(19) United States
(12) Patent Application Publication
(10) Pub. No.: US 2009/0035306 A1
(43) Pub. Date: Feb. 5, 2009

(54) QUINAZOLINONE MODULATORS OF TGR5

(75) Inventors: Anthony B. Pinkerton, San Diego, CA (US); Ayman Kabakibi, San Diego, CA (US); Timothy Z. Hoffman, San Diego, CA (US); Dana L. Siegel, San Diego, CA (US); Stewart A. Noble, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: KALYPSYS, INC., San Diego, CA (US)

(21) Appl. No.: 11/943,097

(22) Filed: Nov. 20, 2007

Related U.S. Application Data
(60) Provisional application No. 60/867,783, filed on Nov. 29, 2006.
(60) Provisional application No. 60/975,561, filed on Sep. 27, 2007.

(51) Int. Cl. C07D 239/88; A61K 31/517; C07D 405/04; C07D 409/04; C07D 401/04; A61K 39/395; A61P 3/00; A61P 3/10; A61P 1/00; A61P 19/02

(52) U.S. Cl. 424/133.1; 544/289; 514/266.31; 514/266.24; 544/284; 514/266.21

(57) ABSTRACT

The present invention relates to quinazolinone compounds useful as modulators of TGR5 and methods for the treatment or prevention of metabolic, cardiovascular, and inflammatory diseases.

This application claims the benefit of priority of U.S. provisional applications No. 60/867,783, filed Nov. 29, 2006, and No. 60/975,561, filed Sep. 27, 2007, the disclosures of which are hereby incorporated by reference as if written herein in their entirety.

Disclosed herein are new quinazolinone compounds and compositions and their application as pharmaceuticals for the treatment of disease. Methods of modulation of TGR5 activity in a human or animal subject are also provided for the treatment diseases such as metabolic, cardiovascular, and inflammatory diseases, for example diabetes, obesity, myocardial infarction, angina pectoris, coronary artery disease, atherosclerosis, cardiac hypertrophy, rheumatoid arthritis, asthma, chronic obstructive pulmonary disease (COPD), psoriasis, ulcerative colitis, allergic diseases, fatty liver, liver fibrosis, kidney fibrosis, anorexia nervosa and bulimia nervosa.

Obesity is a growing threat to the global health by virtue of its association with a cluster of diseases that include insulin resistance, glucose intolerance, dyslipidemia, and hypertension, collectively known as the metabolic syndrome or syndrome X. It is well documented that patients with metabolic syndrome have a higher risk for coronary heart disease and stroke [Grundy S. M. et al. Circulation 112:e285-e290, 2005]. The treatment of obesity will require complex solutions, including increased public awareness to diminish food portions, improved food choices and increased physical activity. However, epidemiologic studies have shown that treating diabetes/insulin resistance in these patients can reduce the risk of coronary artery disease. Marketed drugs to treat diabetes and insulin resistance include biguanides (such as metformin), peroxisome proliferator activated receptor gamma (PPARγ) agonists (such as rosiglitazone and pioglitazone), sulphonylureas, and most recently GLP-1 mimetics such as Exenatide (Byetta). However, there remains a need for additional agents that can perhaps treat the root cause(s) of metabolic syndrome by treating obesity and diabetes. TGR5 modulators described herein might represent such an opportunity.

Bile acids (BA) are amphipathic molecules which are synthesized in the liver from cholesterol and stored in the gall bladder until secretion to the duodenum and intestine to play an important role in the solubilization and absorption of dietary fat and lipid-soluble vitamins. Approx. 99% of BA are absorbed again by passive diffusion and active transport in the terminal ileum and transported back to the liver via the portal vein (enterohepatic circulation). In the liver, BA decrease their own biosynthesis from cholesterol through the activation of the farnesoid X receptor alpha (FXRα) and small heterodimer partner (SHP), leading to the transcriptional repression of cholesterol 7α-hydroxylase, the rate-limiting step of BA biosynthesis from cholesterol.

Recently, two groups independently discovered the GPCR, TGR5 (aka M-BAR) which responds to bile acids [Kawamata Y. et al, J. Biol. Chem., 278:9435-9440, 2003; Maruyama T. et al. Biochem. Biophs. Res. Commun. 298, 714-719, 2002]. TGR5 is a seven transmembrane Gs-coupled GPCR and stimulation by ligand binding causes activation of adenylyl cyclase which leads to the elevation of intracellular cAMP and subsequent activation of downstream signaling pathways. The human receptor shares 86, 90, 82, and 83% amino acid identity to bovine, rabbit, rat, and mouse receptor, respectively. TGR5 is abundantly expressed in the lung, spleen, small intestine, placenta and mononuclear cells (Kawamata Y. et al, J. Biol. Chem., 278:9435-9440, 2003). Bile acids induced receptor internalization, intracellular cAMP production and activation of extracellular signal-regulated kinase in TGR5-expressing HEK293 and CHO cells. In addition, TGR5 was found to be abundantly expressed in monocytes/macrophages from humans and rabbits (Kawamata Y. et al, J. Biol. Chem., 278:9435-9440, 2003), and bile acid treatment suppressed LPS-induced cytokine production in rabbit alveolar macrophages and human THP-1 cells expressing TGR5. These data suggest that bile acids can suppress the macrophage function via activation of TGR5.

Maruyama et al. [Maruyama T. et al. Biochem. Biophs. Res. Commun. 298, 714-719, 2002] showed that TGR5 is expressed in intestinal enteroendocrine cell lines from human (NCI-H716) and murine (STC-1, GLUTag) origin, but not in the intestinal epithelial cells (CaCo-2 and HT-29). Stimulation of TGR5 by BA in NCI-H716 cells stimulated cAMP production. This suggested that bile acids may induce the secretion of glucagon-like peptide-1 (GLP-1) or cholecystokinin (CCK) from the enteroendocrine cells through TGR5 stimulation, since cAMP stimulated the secretion of GLP-1 and CCK from these cells [Reimer R. A. et al. Endocrinology 142, 4522-4528, 2001; Chang C. H. et al. Am. J. Physiol. 271, G516-G523, 1996; Brubaker P. L. et al, Endocrinology 139, 4108-4114, 1998]. This hypothesis was recently confirmed in a publication by Katsuma S. et al. who demonstrated that activation of TGR5 by BA promoted GLP-1 in STC-1 cells [Katsuma S. et al. Biochem. Biophys. Res. Commun. 329, 386-390, 2005]. RNA interference experiments revealed that reduced expression of TGR5 resulted in reduced secretion of GLP-1. GLP-1 has been shown to stimulate insulin release in a glucose dependent manner in humans [Kreymann et al. Lancet 2 (8571) 1300-1304, 1987] and studies in experimental animals demonstrated that this incretin hormone is necessary for normal glucose homeostasis. In addition, GLP-1 can exert several beneficial effects in diabetes and obesity, including 1) increased glucose disposal, 2) suppression in glucose production, 3) reduced gastric emptying, 4) reduction in food intake and 5) weight loss.

Furthermore, recently published data suggested that activation of TGR5 might be beneficial for the treatment of obesity and diabetes. Watanabe et al. (Nature, 439, 484-489, 2006) reported that mice fed high fat diet (HFD) containing 0.5% cholic acid gained less weight than control mice on HFD alone. There was no difference between the two groups in terms of food intake. These effects were independent of FXR-alpha, and instead stem from the binding of bile acids to TGR5 and the subsequent induction of the cAMP-dependent thyroid hormone activating enzyme type 2 (D2) which converts the inactive T3 into the active T4, leading to stimulation of the thyroid hormone receptor and promoting energy expenditure. Mice lacking the D2 gene (D2^{-/-}) were resistant to cholic acid-induced weight loss. In both rodents and humans, the most thermogenically important tissues (the brown adipose and skeletal muscle) are specifically targeted by this mechanism because they co-express D2 and TGR5. The BA-TGR5-cAMP-D2 signaling pathway is therefore a crucial mechanism for fine-tuning energy homeostasis that can be targeted to improve metabolic control.

Taken together, a small molecule TGR5 modulator could be used for the treatment of obesity, diabetes and a wide range of acute and chronic inflammatory diseases.

Recently, certain substituted heterocyclic compounds have been described as agonists of TGR5 for the treatment of metabolic, cardiovascular, and inflammatory diseases (EP01/591120A1, WO04/043468A1, WO04/067008A1, and JP24346059A2). However, as none of these compounds have yet been approved for use in the treatment of disease, a need still exists in the art to provide novel modulators of TGR5.

Novel compounds and pharmaceutical compositions, certain of which have been found to modulate TGR5 have been discovered, together with methods of synthesizing and using the compounds including methods for the treatment of TGR5-mediated diseases in a patient by administering the compounds.

In certain embodiments of the present invention, compounds have structural Formula I:

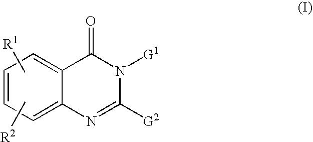

or a salt, ester, or prodrug thereof, wherein:

G¹ is selected from the group consisting of alkyl, alkenyl, alkynyl, aryl, heteroaryl, cycloalkyl, heterocycloalkyl, arylalkyl, heteroarylalkyl, cycloalkylalkyl, arylamino, heteroarylamino, cycloalkylamino, heterocycloalkylamino, arylalkylamino, arylalkenylamino, heteroarylalkylamino, and cycloalkylalkylamino, any of which may be optionally substituted;

G² is selected from the group consisting of alkyl, alkenyl, alkynyl, aryl, heteroaryl, cycloalkyl, heterocycloalkyl, aminoalkyl, acylaminoalkyl, alkylthio, amidoalkylthio, amidoalkylthioalkylthio, -alkyl-N(acyl)arylalkyl, -alkyl-N(acyl)heteroarylalkyl, -alkyl-N(acyl)alkyl, -alkyl-N(arylamido)alkyl, -alkyl-N(arylheteroalkylcarbonyl)arylalkyl, -alkyl-N(arylheteroalkylcarbonyl)alkyl, and —CH(R³)N(R⁴)COR⁵, any of which may be optionally substituted; or G¹ and G², together with the atoms to which they are attached, may be joined to form an optionally substituted heterocycloalkyl moiety;

R¹ and R² are independently selected from the group consisting of hydrogen, halogen, amino, cyano, nitro, hydroxy, hydroxyalkyl, alkoxy, alkyl, acyl, alkenyl, alkynyl, alkoxyalkyl, alkylamino, alkylaminoalkyl, alkylcarbonyl, aminoalkyl, carboxyl, alkoxycarbonyl, alkylthio, alkylsulfonyl, aryl, arylalkyl, arylalkenyl, arylalkynyl, cycloalkyl, cycloalkylalkyl, haloalkyl, perhaloalkyl, perhaloalkoxy, heteroaryl, heteroarylalkyl, heteroarylalkenyl, and heterocycloalkyl, any of which may be optionally substituted;

R³ is selected from the group consisting of hydrogen, alkyl, alkenyl, alkynyl, aryl, heteroaryl, heterocycloalkyl, cycloalkyl, and cycloalkylalkyl, any of which may be optionally substituted;

R⁴ is selected from the group consisting of hydrogen, alkyl, alkenyl, alkynyl, aryl, heteroaryl, heterocycloalkyl, cycloalkyl, cycloalkylalkyl, arylalkyl, heteroarylalkyl, alkoxyalkyl, and alkylthioalkyl, any of which may be optionally substituted; and

R⁵ is selected from the group consisting of aryl, arylalkyl, aryloxyalkyl, alkyl, arylalkyloxyalkyl, arylalkenyl, arylamino, cycloalkyl, cycloalkylalkyl, heterocycloalkyl, heteroaryl, heteroarylalkyl, heteroaryloxyalkyl, heteroalkyl, heteroarylalkyloxyalkyl, heteroarylalkenyl, and heteroarylamino, any of which may be optionally substituted.

Certain compounds disclosed herein may possess useful TGR5 modulating activity, and may be used in the treatment or prophylaxis of a disease or condition in which TGR5 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for modulating TGR5. Other embodiments provide methods for treating a TGR5-mediated disorder in a patient in need of such treatment, comprising administering to said patient a therapeutically effective amount of a compound or composition as disclosed herein. Also provided is the use of certain compounds disclosed herein for use in the manufacture of a medicament for the treatment of a disease or condition ameliorated by the modulation of TGR5.

In certain embodiments, a method of treatment of a TGR5-mediated disease comprises the administration, to a patient in need thereof, of a therapeutically effective amount of a compound having structural Formula II:

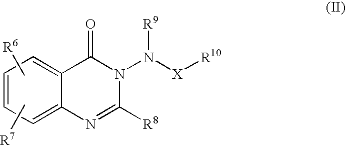

or a salt, ester, or prodrug thereof, wherein:

X is C₁₋₂alkyl, optionally substituted with one or more substituents selected from the group consisting of C₁₋₃alkyl, halogen, and oxo;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl, and amino; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

In further embodiments, R⁶ and R⁷ are independently selected from the group consisting of hydrogen, halogen, alkoxy, alkyl, haloalkyl, perhaloalkyl, and perhaloalkoxy; R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; R⁹ is selected from the group consisting of hydrogen and lower alkyl; and R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments, X is CH₂; R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; R⁹ is hydrogen; and R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments, R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments, R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

In yet further embodiments, R¹⁰ is phenyl, which is substituted in the para position by halogen.

In yet further embodiments, said compound having structural Formula II is selected from the group consisting of Examples 1 to 119.

In yet further embodiments, said disease is a metabolic disease.

In yet further embodiments, said disease is selected from the group consisting of inadequate glucose tolerance, insulin resistance, type I diabetes, and type II diabetes.

In yet further embodiments, the method further comprises the administration of another therapeutic agent.

In yet further embodiments, said agent is selected from the group consisting of insulin, metformin, Glipizide, glyburide, Amaryl, gliclazide, meglitinides, nateglinide, repaglinide, pramlintide, PTP-112, SB-517955, SB-4195052, SB-216763, NN-57-05441, NN-57-05445, GW-0791, AGN-¹⁹4²⁰4, T-1095, BAY R3401, acarbose, miglitol, voglibose, Exendin-4, DPP728, LAF237, vildagliptin, BMS477118, PT-100, GSK-823093, PSN-9301, T-6666, SYR-322, SYR-619, Liraglutide, CJC-1134-PC, naliglutide, MK-0431, saxagliptin, GSK23A, pioglitazone, rosiglitazone, AVE2268, GW869682, GSK189075, APD668, PSN-119-1, PSN-821, rosuvastatin, atrovastatin, simvastatin, lovastatin, pravastatin, fluvastatin, cerivastatin, rosuvastatin, pitavastatin, fenofibrate, benzafibrate, clofibrate, gemfibrozil, Ezetimibe, eflucimibe, CP-529414, CETi-1, JTT-705, cholestyramine, colestipol, niacin, implitapide, (R)-1-{4-[5-methyl-2-(4-trifluoromethyl-phenyl)-oxazol-4-ylmethoxy]-benzenesulfonyl}2,3-dihydro-1H-indole-2-carboxylic acid, and GI-262570.

In yet further embodiments, said disease is associated with perturbed bile acid metabolism.

In yet further embodiments, the method further comprises the administration of another therapeutic agent.

In yet further embodiments, said disease is an inflammatory disease.

In yet further embodiments, said disease is selected from the group consisting of rheumatoid arthritis, ulcerative colitis, and inflammatory bowel disease.

In yet further embodiments, the method further comprises the administration of another therapeutic agent.

In yet further embodiments, said agent is selected from the group consisting of betamethasone dipropionate, betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream, guaifenesin, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin, clonidine, codeine, loperamide, tramadol, morphine, fentanyl, oxycodone, hydrocodone, levorphanol, butorphanol, menthol, oil of wintergreen, camphor, eucalyptus oil, turpentine oil, acetaminophen, infliximab, etanerecept, infliximab, and capsaicin.

In yet further embodiments, said disease is obesity.

In yet further embodiments, said method achieves an effect selected from the group consisting of decreasing body weight and controlling weight gain.

In yet further embodiments, the method further comprises the administration of another therapeutic agent.

In yet further embodiments, said agent is selected from the group consisting of sibutramine, bromocriptine, Orlistat, rimonabant, Axokine, and bupropion.

In further embodiments, a pharmaceutical composition comprises a pharmaceutically acceptable carrier together with a compound having structural Formula II:

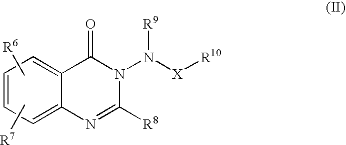

or a salt, ester, or prodrug thereof, wherein:

X is CH₂;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and

R¹⁰ is selected from the group consisting of aryl, heteroaryl, cycloalkyl, and heterocycloalkyl, substituted with at least one substituent selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

In yet further embodiments R⁶ and R⁷ are independently selected from the group consisting of hydrogen, halogen, alkoxy, alkyl, haloalkyl, perhaloalkyl, and perhaloalkoxy; R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; R⁹ is selected from the group consisting of hydrogen and lower alkyl; and R¹⁰ is selected from the group consisting of aryl, heteroaryl, cycloalkyl, and heterocycloalkyl, substituted with at least one substituent selected from the group consisting of halogen, alkyl, haloalkyl, perhaloalkyl, alkoxy, perhaloalkoxy, carboxyl, and cyano.

In yet further embodiments R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; R⁹ is hydrogen; and R¹⁰ is selected from the group consisting of lower aryl, lower heteroaryl, lower cycloalkyl, and lower heterocycloalkyl, substituted with at least one substituent selected from the group consisting of halogen, alkyl, haloalkyl, perhaloalkyl, alkoxy, perhaloalkoxy, carboxyl, and cyano.

In yet further embodiments R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of phenyl, 5-membered monocyclic heteroaryl, and 6-membered monocyclic heteroaryl, substituted with at least one substituent selected from the group consisting of halogen, alkyl, haloalkyl, perhaloalkyl, alkoxy, perhaloalkoxy, carboxyl, and cyano.

In yet further embodiments R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is phenyl, substituted with at least one substituent selected from the group consisting of halogen, alkyl, haloalkyl, perhaloalkyl, alkoxy, perhaloalkoxy, carboxyl, and cyano.

In yet further embodiments R¹⁰ is phenyl, which is substituted in the para position by halogen.

In further embodiments, a pharmaceutical composition comprises a pharmaceutically acceptable carrier together with a compound having structural

### Formula II:

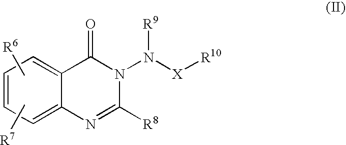

or a salt, ester, or prodrug thereof, wherein:

X is C₁₋₂alkyl, optionally substituted with one or more substituents selected from the group consisting of C₁₋₃alkyl, halogen, and oxo;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of bicyclic aryl, 5-membered nitrogen-containing heteroaryl, 6-membered heteroaryl, bicyclic heteroaryl, saturated or partially unsaturated heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl, and amino; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

In yet further embodiments X is CH₂; R⁶ and R⁷ are independently selected from the group consisting of hydrogen, halogen, alkoxy, alkyl, haloalkyl, perhaloalkyl, and perhaloalkoxy; R⁸ is selected from the group consisting of bicyclic aryl, 5-membered nitrogen-containing heteroaryl, 6-membered heteroaryl, bicyclic heteroaryl, saturated or partially unsaturated heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; R⁹ is selected from the group consisting of hydrogen and lower alkyl; and R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is selected from the group consisting of bicyclic aryl, 5-membered nitrogen-containing heteroaryl, 6-membered heteroaryl, bicyclic heteroaryl, lower saturated or partially unsaturated heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; R⁹ is hydrogen; and R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is selected from the group consisting of 5-membered nitrogen-containing monocyclic heteroaryl and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

In yet further embodiments R¹⁰ is phenyl, which is substituted in the para position by halogen.

In further embodiments, a pharmaceutical composition comprises a pharmaceutically acceptable carrier together with a compound having structural Formula II:

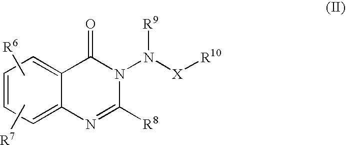

or a salt, ester, or prodrug thereof, wherein:

X is CH₂;

R⁶ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁷ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is hydrogen; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two substituents may be joined together to form a fused five-, six-, or seven-membered carbocyclic or heterocyclic ring comprising zero to three heteroatoms.

In yet further embodiments R⁶ is selected from the group consisting of halogen, alkoxy, alkyl, haloalkyl, perhaloalkyl, and perhaloalkoxy; R⁷ is selected from the group consisting of hydrogen, halogen, alkoxy, alkyl, haloalkyl, perhaloalkyl, and perhaloalkoxy; R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

In yet further embodiments R¹⁰ is phenyl, which is substituted in the para position by halogen.

In further embodiments a pharmaceutical composition comprises a pharmaceutically acceptable carrier together with a compound selected from the group consisting of Examples 1 to 119.

In further embodiments, a compound has structural Formula II:

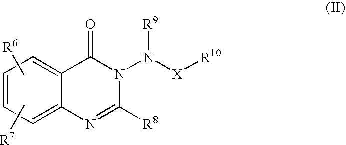

or a salt, ester, or prodrug thereof, wherein:

X is CH₂;

R⁶ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁷ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is hydrogen; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

In yet further embodiments R⁶ is selected from the group consisting of halogen, alkoxy, alkyl, haloalkyl, perhaloalkyl, and perhaloalkoxy; R⁷ is selected from the group consisting of hydrogen, halogen, alkoxy, alkyl, haloalkyl, perhaloalkyl, and perhaloalkoxy; R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

In yet further embodiments R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

In yet further embodiments R¹⁰ is phenyl, which is substituted in the para position by halogen.

In further embodiments, a compound has structural Formula II:

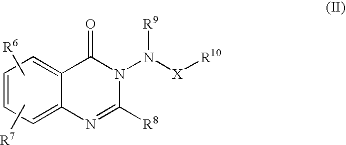

or a salt, ester, or prodrug thereof, wherein:

X is CH₂;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and

R¹⁰ is para-chlorophenyl.

In yet further embodiments R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R⁹ is selected from the group consisting of hydrogen and lower alkyl.

In yet further embodiments R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and R⁹ is hydrogen.

In yet further embodiments R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino.

In yet further embodiments R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino. In further embodiments, a compound is selected from the group consisting of Examples 7 to 119.

In further embodiments a compound for use as a medicament has structural Formula II:

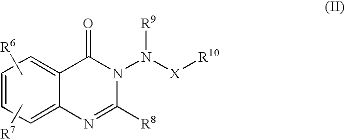

or a salt, ester, or prodrug thereof, wherein:

X is C₁₋₂alkyl, optionally substituted with one or more substituents selected from the group consisting of C₁₋₃alkyl, halogen, and oxo;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising of zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

In further embodiments a compound for use in the manufacture of a medicament for the prevention or treatment of a disease or condition ameliorated by the modulation of TGR5 has structural Formula II:

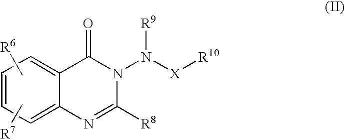

or a salt, ester, or prodrug thereof, wherein:

X is C₁₋₂alkyl, optionally substituted with one or more substituents selected from the group consisting of C₁₋₃alkyl, halogen, and oxo;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

In further embodiments a method of modulation of TGR5 comprises contacting TGR5 with a compound having structural Formula II:

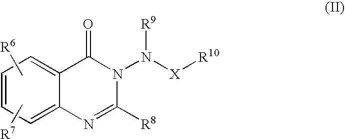

or a salt, ester, or prodrug thereof, wherein:

X is C₁₋₂alkyl, optionally substituted with one or more substituents selected from the group consisting of C₁₋₃alkyl, halogen, and oxo;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

In further embodiments a method for achieving an effect in a patient comprises the administration of a therapeutically effective amount of a compound having structural Formula II:

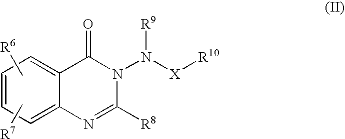

or a salt, ester, or prodrug thereof, wherein:

X is C₁₋₂alkyl, optionally substituted with one or more substituents selected from the group consisting of C₁₋₃alkyl, halogen, and oxo;

R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;

R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;

R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and

R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms; to a patient, wherein the effect is selected from the group consisting of improving glucose tolerance, decreasing insulin resistance, decreasing body weight, controlling weight gain, modulation of type I diabetes, modulation of type II diabetes, modulation of perturbed bile acid metabolism, modulation of rheumatoid arthritis, modulation of ulcerative colitis, and modulation of inflammatory bowel disease.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group—with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S—group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen. The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R′ or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“TGR5 modulator” is used herein to refer to a compound that exhibits an EC₅₀ with respect to TGR5 activity of no more than about 100 μM and more typically not more than about 50 μM, as measured in the cAMP production assay and glucagon-like peptide-1 (GLP-1) secretion assays described generally hereinbelow. “EC₅₀” is that concentration of modulator which either activates or reduces the activity of an enzyme (e.g., (TGR5)) to half-maximal level. Certain compounds disclosed herein have been discovered to exhibit modulatory activity against TGR5. In certain embodiments, compounds will exhibit an EC₅₀ with respect to TGR5 of no more than about 10 μM; in further embodiments, compounds will exhibit an EC₅₀ with respect to TGR5 of no more than about 5 μM; in yet further embodiments, compounds will exhibit an EC₅₀ with respect to TGR5 of not more than about 1 μM; in yet further embodiments, compounds will exhibit an EC₅₀ with respect to TGR5 of not more than about 200 nM, as measured in the TGR5 assay described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with agents found in the following pharmacotherapeutic classifications as indicated below. These lists should not be construed to be closed, but should instead serve as illustrative examples common to the relevant therapeutic area at present. Moreover, combination regimens may include a variety of routes of administration and should include oral, intravenous, intraocular, subcutaneous, dermal, and inhaled topical.

For the treatment of metabolic disorders, compounds according to the present invention may be administered with an agent selected from the group comprising: insulin, insulin derivatives and mimetics, insulin secretagogues, insulin sensitizers, biguanide agents, alpha-glucosidase inhibitors, insulinotropic sulfonylurea receptor ligands, meglitinides, protein tyrosine phosphatase-1B (PTP-1B) inhibitors, GSK3 (glycogen synthase kinase-3) inhibitors, GLP-1 (glucagon like peptide-1), GLP-1 analogs, DPPIV (dipeptidyl peptidase IV) inhibitors, RXR ligands, sodium-dependent glucose co-transporter (SGLT2) inhibitors, glycogen phosphorylase A inhibitors, an AGE breaker, PPAR modulators, non-glitazone type PPARδ agonist, HMG-CoA reductase inhibitors, cholesterol-lowering drugs and anti-obesity agents.

For the treatment of metabolic disorders, compounds according to the present invention may be administered with an agent selected from the group comprising: insulin, metformin, Glipizide, glyburide, Amaryl, gliclazide, meglitinides, nateglinide, repaglinide, amylin mimetics (for example, pramlintide), PTP-112, SB-517955, SB-4195052, SB-216763, N,N-57-05441, N,N-57-05445, GW-0791, AGN-¹⁹4²⁰4, T-1095, BAY R3401, acarbose, miglitol, voglibose, Exendin-4, DPP728, LAF237, vildagliptin, BMS477118, PT-100, GSK-823093, PSN-9301, T-6666, SYR-322, SYR-619, Liraglutide, CJC-1134-PC, naliglutide, MK-0431, saxagliptin, GSK23A, pioglitazone, rosiglitazone, AVE2268, GW869682, GSK189075, GPR119 agonists including, but not limited to APD668, PSN-119-1 and PSN-821, HMG-CoA reductase inhibitors (for example, rosuvastatin, atrovastatin, simvastatin, lovastatin, pravastatin, fluvastatin, cerivastatin, rosuvastatin, pitavastatin and like), cholesterol-lowering drugs (for example, fibrates which include: fenofibrate, benzafibrate, clofibrate, gemfibrozil and like; cholesterol absorption inhibitors such as Ezetimibe, eflucimibe and like compounds), cholesterol ester transfer protein inhibitors (for example, CP-529414, CETi-1, JTT-705 and like compounds), bile acid sequestrants (for example, cholestyramine, colestipol, and like compounds), niacin, microsomal triglyceride transfer protein inhibitors (for example, implitapide), insulin signaling pathway modulators, like inhibitors of protein tyrosine phosphatases (PTPases) and inhibitors of glutamine-fructose-6-phosphate amidotransferase (GFAT), inhibitors of glucose-6-phosphatase (G6 Pase), inhibitors of fructose-1,6-bisphosphatase (F-1,6-BPase), inhibitors of glycogen phosphorylase, glucagon receptor antagonists, inhibitors of phosphoenolpyruvate carboxylase (PEPCK), inhibitors of pyruvate dehydrogenase kinase, activators AMP-activated protein kinase (AMPK), (R)-1-{4-[5-methyl-2-(4-trifluoromethyl-phenyl)-oxazol-4-ylmethoxy]-benzenesulfonyl}2,3-dihydro-1H-indole-2-carboxylic acid described in the patent application WO 03/043985, as compound 19 of Example 4, and GI-262570.

For the treatment of obesity, compounds according to the present invention may be administered with an agent selected from the group comprising: cholescystokinin-A (CCK-A) agonists, serotonin and norepinephrine reuptake inhibitors (for example sibutramine), dopamine agonists (for example, bromocriptine and like) sympathomimetic agents, β3 adrenergic receptor agonists, leptin, leptin analogues, leptin receptor agonists, galanin antagonists, lipase inhibitors (for example Orlistat), Neuropeptide-Y antagonists, glucocorticoid receptor agonists or antagonists, cannabinoid 1 receptor antagonists (for example, rimonabant and like), ciliary neurotropic factors (CNTF, for example Axokine), human agouti-related proteins (AGRP), ghrelin receptor antagonists, histamine 3 receptor antagonists, appetite suppressants (for example, bupropion), urocortin binding protin antagonists, orexin receptor antagonists, and bombesin agonists.

For the treatment of inflammatory diseases, compounds according to the present invention may be administered with an agent selected from the group comprising: corticosteroids, non-steroidal anti-inflammatories, muscle relaxants and combinations thereof with other agents, anaesthetics and combinations thereof with other agents, expectorants and combinations thereof with other agents, antidepressants, anticonvulsants and combinations thereof, antihypertensives, opioids, topical cannabinoids, and other agents, such as capsaicin.

For the treatment of inflammatory diseases, compounds according to the present invention may be administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream (Eutectic Mixture of Local Anesthetics (lidocaine 2.5% and prilocalne 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab; n) nitric oxide synthase inhibitors, particularly inhibitors of inducible nitric oxide synthase; anti-TNFα agents including, but not limited to etanerecept and infliximab, and other agents, such as capsaicin.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating TGR5-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of TGR5-mediated disorders.

Specific diseases to be treated by the compounds, compositions, and methods disclosed herein include: diabetes (type I and type II) and conditions associated with diabetic diseases which include, but are not limited to, hyperglycemia, hyperlipidemia, hyperinsulinemia, insulin resistance, inadequate glucose tolerance, impaired glucose metabolism, diabetic nephropathy, glomerulosclerosis, diabetic neuropathy, erectile dysfunction, macular degeneration, diabetic retinopathy, chronic microvascular complications, peripheral vascular disease, cataracts, stroke, foot ulcerations, renal failure, kidney disease, ketosis, metabolic acidosis, and related disorders, obesity, myocardial infarction, angina pectoris, coronary artery disease, atherosclerosis, cardiac hypertrophy, allergic diseases, fatty liver disease, nonalcoholic steatohepatitis, liver fibrosis, kidney fibrosis, anorexia nervosa, bulimia vervosa, autoimmune diseases, inflammatory diseases including rheumatoid arthritis, asthma, chronic obstructive pulmonary disease (COPD), psoriasis, ulcerative colitis, proliferative disorders, infectious diseases, angiogenic disorders, reperfusion/ischemia in stroke, vascular hyperplasia, organ hypoxia, cardiac hypertrophy, thrombin-induced platelet aggregation, and conditions associated with prostaglandin endoperoxidase synthetase-2 (COX-2).

In certain aspects of the invention, the disease is obesity and the effects to be achieved in a human or animal patient include decreasing body weight and controlling weight gain.

In certain aspects of the invention, the disease is associated with perturbed bile acid metabolism, including, but not limited to gall bladder stones, cholecystitis, cholangitis, choledocholithiasis, jaundice, and obstetric cholestasis and the itch associated with it.

In certain aspects of the invention, the disease is a hyperproliferative condition of the human or animal body, including, but not limited to restenosis, inflammation, immune disorders, cardiac hypertrophy, atherosclerosis, pain, migraine, angiogenesis-related conditions or disorders, proliferation induced after medical conditions, including but not limited to surgery, angioplasty, or other conditions.

The compositions of the present invention are useful as anti-inflammatory agents with the additional benefit of having significantly less harmful side effects. The compositions are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compositions may also be used in the treatment of pulmonary inflammation, such as that associated with viral infections and cystic fibrosis. The invention further extends to the particular inflammatory disease rheumatoid arthritis.

Further inflammatory diseases which may be prevented or treated include, without limitation: asthma, allergies, respiratory distress syndrome or acute or chronic pancreatitis. Furthermore, respiratory system diseases may be prevented or treated including but not limited to chronic obstructive pulmonary disease, pulmonary fibrosis, ulcerative colitis, inflammatory bowel disease, Crohn's disease, peptic ulceration, gastritis, psoriasis, and skin inflammation.

In some aspects of the invention, the disease to be treated by the methods of the present invention may be an opthalmologic disorder. Opthalmologic diseases and other diseases in which angiogenesis plays a role in pathogenesis, may be treated or prevented and include, without limitation, dry eye (including Sjögren's syndrome), macular degeneration, closed and wide angle glaucoma, retinal ganglion degeneration, occular ischemia, retinitis, retinopathies, uveitis, ocular photophobia, and of inflammation and pain associated with acute injury to the eye tissue. The compositions can be used to treat glaucomatous retinopathy and/or diabetic retinopathy. The compositions can also be used to treat post-operative inflammation or pain as from ophthalmic surgery such as cataract surgery and refractive surgery.

In some aspects of the invention, the disease to be treated by the methods of the present invention may be an autoimmune disease. Autoimmune diseases which may be prevented or treated include, but are not limited to: rheumatoid arthritis, inflammatory bowel disease, inflammatory pain, ulcerative colitis, Crohn's disease, periodontal disease, temporomandibular joint disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, chronic active hepatitis, myasthenia gravis, atopic dermatitis, graft vs. host disease, and psoriasis. Inflammatory diseases which may be prevented or treated include, but are not limited to: asthma, allergies, respiratory distress syndrome or acute or chronic pancreatitis. The invention further extends to the particular autoimmune disease rheumatoid arthritis.

Metabolic diseases which may be treated or prevented include, without limitation, metabolic syndrome, insulin resistance, and Type 1 and Type 2 diabetes. In addition, the compositions of the subject invention can be used to treat insulin resistance and other metabolic disorders such as atherosclerosis that are typically associated with an exaggerated inflammatory signaling.

The compositions of the present invention are also useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, swelling occurring after injury, ischemias including myocardial ischemia, cardiovascular ischemia, and ischemia secondary to cardiac arrest, and the like. These compositions can also be used to treat allergic rhinitis, respiratory distress syndrome, endotoxin shock syndrome, and atherosclerosis.

In some aspects of the invention, the disease to be treated by the methods of the present invention may be a cardiovascular condition. In certain embodiments, said cardiovascular condition is selected from the group consisting of atherosclerosis, cardiac hypertrophy, idiopathic cardiomyopathies, heart failure, angiogenesis-related conditions or disorders, and proliferation induced after medical conditions, including, but not limited to restenosis resulting from surgery and angioplasty.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### General Synthetic Methods for Preparing Compounds

The following schemes can be used to practice the present invention. Starting materials are commercially available, made by known procedures, or prepared as illustrated herein.

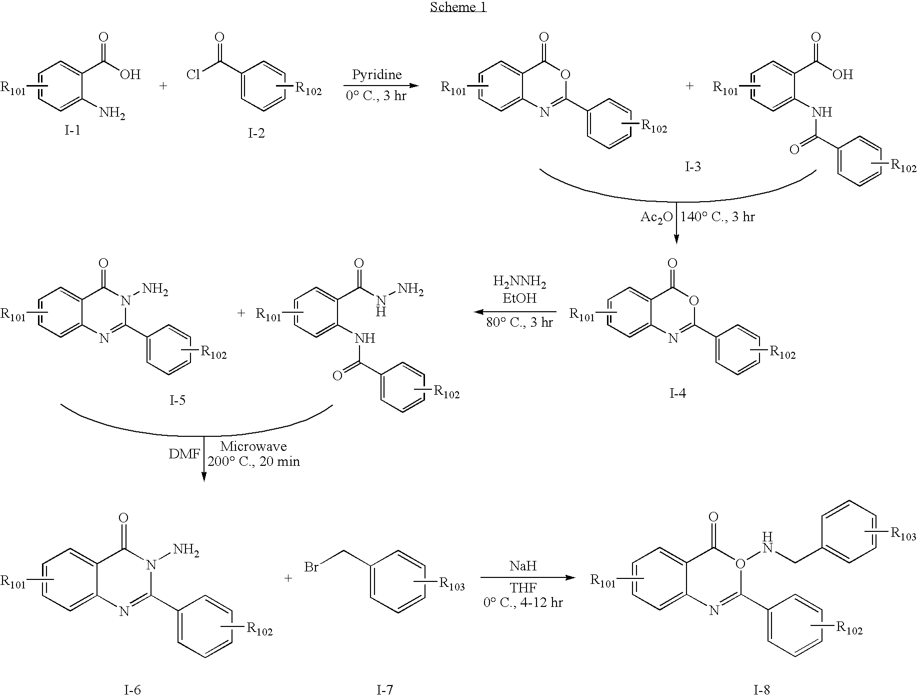

Examples 1-92 and 103-119 can be synthesized using the general synthetic procedure set forth in Scheme I: An appropriately substituted anthranilic acid I-1 and acid chloride I-2 are reacted in pyridine to give a mixture of amides and cyclized products I-3. Heating the mixture in acetic anhydride gives cyclized lactone I-4. Reaction of lactones I-4 with hydrazine in refluxing ethanol gives hydrazides I-5, which were further reacted in DMF at elevated temperatures in a microwave to give hydrazides I-6. Hydrazides I-6 are then reacted with an appropriately substituted benzyl bromide I-7 under standard conditions to give desired quinazolinones I-8.

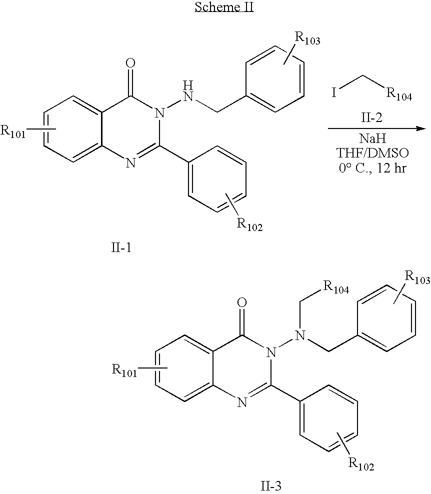

Example 93 can be synthesized using the general synthetic procedure set forth in Scheme II: An appropriately substituted quinazolinone II-1, is alkylated with iodide II-2 to give desired products II-3.

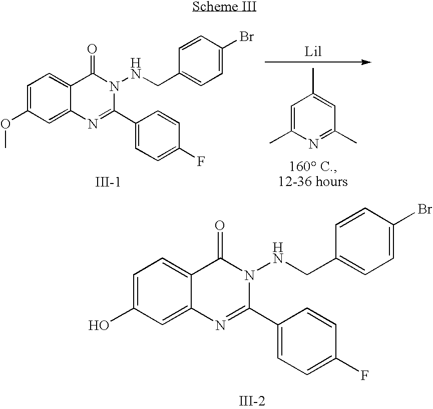

Examples 94-100 can be synthesized using the general synthetic procedure set forth in Scheme III: An appropriately substituted methoxy quinazolinone III-1 is dealkylated with lithium iodide to give desired product III-2.

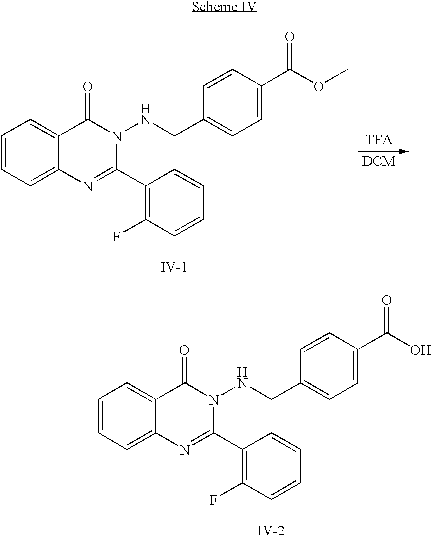

Example 101 can be synthesized using the general synthetic procedure set forth in Scheme IV: An appropriately substituted ester quinazolinone IV-1 is deprotected with acid to give desired product IV-2.

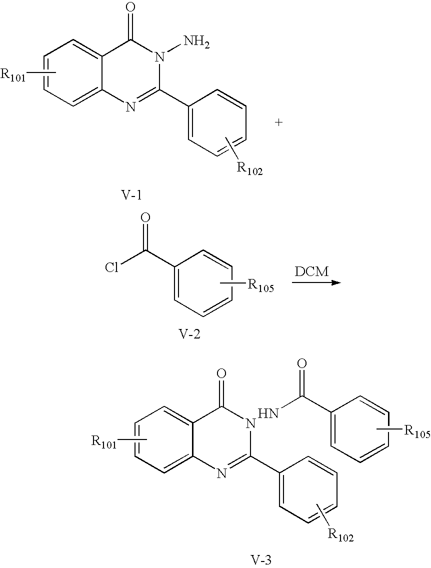

Example 102 can be synthesized using the general synthetic procedure set forth in Scheme V: An appropriately substituted hydrazide V-1 is reacted with an appropriate acid chloride V-2 to give desired product V-3.

The invention is further illustrated by the following examples.

### EXAMPLE 1

### 3-(4-bromobenzylamino)-2-(4-fluorophenyl)quinazolin-4(3H)-one)

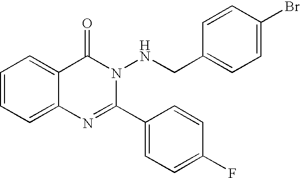

### Step 1

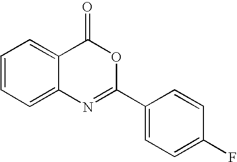

2-(4-Fluorophenyl)-4H-benzo[d][1,3]oxazin-4-one): In a 40 mL septa sealed vial purged with nitrogen, anthranilic acid (500 mg, 3.65 mmol) was stirred in 10 mL of anhydrous pyridine in an ice bath (0° C.) for 10 minutes. 4-Fluorobenzoyl chloride (440 μL, 3.72 mmol) was then carefully added and stirred for three hours gradually warming to room temperature. Hexane (50 mL) was added to the mixture and the precipitate filtered, then rinsed three additional times with 20 mL of hexane. The filtrate was then poured into 100 mL of water and extracted three times with 50 mL of ethyl acetate. The filtered solids were added to the combined organic layers and concentrated to give approximately 550 mg (61%) of cyclized 2-(4-fluorophenyl)-4H-benzo[d][1,3]oxazin-4-one) and uncyclized (2-(4-fluorobenzamido)benzoic acid. The mixture of 2-(4-fluorophenyl)-4H-benzo[d][1,3]oxazin-4-one) and uncyclized (2-(4-fluorobenzamido)benzoic acid was stirred in acetic anhydride (5 mL) in a 20 mL septa sealed vial and heated to 140° C. for three hours, then concentrated under a stream of nitrogen to give 2-(4-fluorophenyl)-4H-benzo[d][1,3]oxazin-4-one.

### Step 2

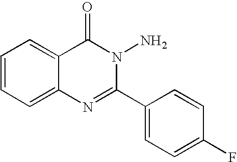

3-amino-2-(4-fluorophenyl)quinazolin-4(3H)-one: In a 20 mL septa sealed vial, 2-(4-fluorophenyl)-4H-benzo[d][1,3]oxazin-4-one (500 mg, 2.07 mmol) was stirred in 10 mL of absolute ethanol. Anhydrous hydrazine (325 μL) was added and the reaction heated to 80° C. for three hours. The reaction was then cooled to 40° C. and concentrated under a stream of nitrogen to give approximately 265 mg (48%) of the cyclized (3-amino-2-(4-fluorophenyl)quinazolin-4(3H)-one) and uncyclized (4-fluoro-N-(2-(hydrazinecarbonyl)phenyl)benzamide). The mixture (200 mg) was transferred to a 2 mL microwave tube with 1 mL of anhydrous DMF. The tube was sealed and the mixture microwaved at 200° C. for 20 minutes. The reaction was then poured into 5 mL of water and extracted three times with ethyl acetate. The organic layers were combined, washed once with brine, dried over anhydrous sodium sulfate, filtered and concentrated to give 98% cyclized product 3-amino-2-(4-fluorophenyl)quinazolin-4(3H)-one.

### Step 3

3-(4-bromobenzylamino)-2-(4-fluorophenyl)quinazolin-4(3H)-one): In a 20 mL septa sealed vial, 3-amino-2-(4-fluorophenyl)quinazolin-4(3H)-one (200 mg, 0.78 mmol) was stirred in 5 mL of anhydrous THF in an ice bath (0° C.) for 10 minutes. Sodium hydride (62 mg, 1.56 mmol, 60% in oil) was carefully added and stirred at 0° C. for five minutes. Then 4-bromobenzyl bromide (292 mg, 1.17 mmol) was added and the reaction stirred for 12 hours gradually warming to room temperature. The reaction was poured into 5 mL of water and extracted three times with ethyl acetate. The organic layers were combined, washed once with brine, dried over anhydrous sodium sulfate, filtered and concentrated. Hexane was then added to the crude material and pure 3-(4-bromobenzylamino)-2-(4-fluorophenyl)quinazolin-4(3H)-one was filtered off as a white solid.

¹H-NMR: (CDCl₃) 8.32 (d, 1H), 7.80 (d, 2H), 7.78 (d, 2H), 7.54 (dd, 1H), 7.32 (d, 2H), 7.81 (dd, 2H), 6.82 (d, 2H), 6.02 (t, 1H), 3.80 (brd, 2H) ppm

MS: (424.3)

### EXAMPLE 2

### 2-(4-fluorophenyl)-3-(4-isopropylbenzylamino)quinazolin-4(3H)-one

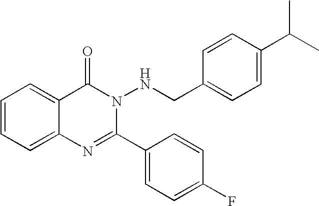

Prepared as described in Example 1.

¹H-NMR: (CDCl₃) 8.34 (d, 1H), 7.84 (dd, 2H), 7.79 (dd, 2H), 7.51 (dd, 1H), 7.13 (dd, 2H), 7.09 (dd, 2H), 6.91 (d, 2H), 6.02 (t, 1H), 3.79 (brd, 2H), 2.82 (m, 1H), 1.22 (dd, 6H) ppm

MS: (387.5)

### EXAMPLE 3

### 3-(3-bromobenzylamino)-2-(2-fluorophenyl)quinazolin-4(3H)-one

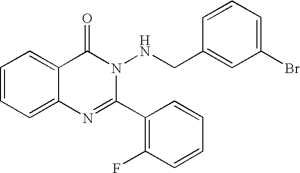

Prepared as described in Example 1.

¹H-NMR: (CDCl₃) 8.39 (d, 1H), 7.82 (dd, 2H), 7.58 (dd, 2H), 7.38 (dd, 2H), 7.24 (d, 1H), 7.20 (d, 1H), 7.02 (t, 1H), 6.89 (s, 1H), 6.80 (d, 1H), 5.80 (t, 1H), 3.92 (brd, 2H) ppm

MS: (424.3)

### EXAMPLE 4

### 3-(2,4-dichlorobenzylamino)-2-(2-fluorophenyl)quinazolin-4(3H)-one

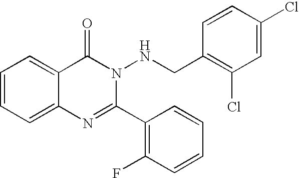

Prepared as described in Example 1.

¹H-NMR: (CDCl₃) 8.18 (d, 1H), 7.80 (d, 1H), 7.78 (d, 1H), 7.58 (dd, 1H), 7.42 (dd, 2H), 7.26 (s, 1H), 7.18 (dd, 1H), 7.16 (s, 1H), 7.02 (dd, 1H), 6.80 (d, 1H), 5.88 (t, 1H), 4.02 (brd, 2H) ppm

MS: (414.3)

### EXAMPLE 5

### 3-(4-bromobenzylamino)-2-(2-fluorophenyl)quinazolin-4(3H)-one

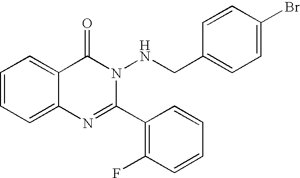

Prepared as described in Example 1.

¹H-NMR: (CDCl₃) 8.19 (d, 1H), 7.82 (dd, 1H), 7.78 (dd, 1H), 7.58 (dd, 1H), 7.54 (dd, 1H), 7.39 (dd, 1H), 7.26 (d, 1H), 7.24 (d, 1H), 7.16 (dd, 2H), 6.71 (d, 2H), 5.80 (t, 1H), 3.90 (brd, 2H) ppm

MS: (424.3)

### EXAMPLE 6

### 3-(benzylamino)-2-phenylquinazolin-4(3H)-one

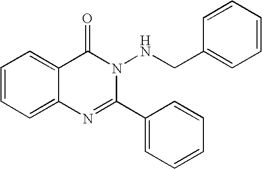

Prepared as described in Example 1.

¹H-NMR: (CDCl₃) 8.38 (d, 1H), 7.82 (m, 4H), 7.56 (m, 2H), 7.54 (m, 2H), 7.23 (d, 1H), 7.20 (m, 2H), 6.99 (d, 2H), 6.02 (t, 1H), 3.82 (brd, 2H) ppm

MS: (327.4)

### EXAMPLE 7

### 3-(2,4-difluorobenzylamino)-2-(3-fluorophenyl)quinazolin-4(3H)-one

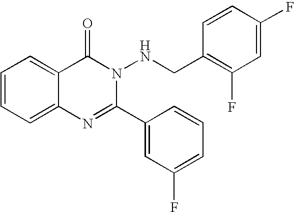

Prepared as described in Example 1.

¹H-NMR: (CDCl₃) 8.31 (d, 1H), 7.82 (m, 2H), 7.58 (m, 2H), 7.60 (m, 1H), 7.38 (m, 1H), 7.20 (dd, 1H), 6.88 (dd, 1H), 6.63 (m, 1H), 6.00 (m, 1H), 6.05 (t, 1H), 3.92 (brd, 2H) ppm

MS: (381.4)

### EXAMPLE 8

### 3-(4-bromobenzylamino)-2-(2(trifluoromethyl)phenyl)quinazolin-4(3H)-one

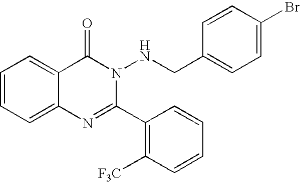

Prepared as described in Example 1.

¹H-NMR: (CDCl₃) 8.39 (d, 1H), 7.82 (dd, 2H), 7.78 (d, 1H), 7.64 (dd, 2H), 7.62 (d, 1H), 7.42 (d, 1H), 7.26 (d, 2H), 6.71 (d, 2H), 5.51 (t, 1H), 4.01 (brd, 1H), 3.78 (brd, 1H) ppm

MS: (474.3)

### EXAMPLE 9

### 3-(4-bromobenzylamino)-2-(4-(trifluoromethyl)phenyl)quinazolin-4(3H)-one

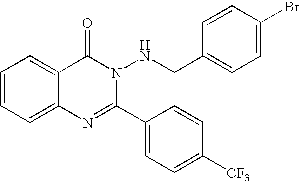

Prepared as described in Example 1.

¹H-NMR: (CDCl₃) 8.38 (d, 1H), 7.82 (dd, 2H), 7.80 (dd, 2H), 7.69 (d, 2H), 7.58 (dd, 1H), 7.23 (d, 2H), 6.77 (d, 2H), 6.04 (t, 1H), 3.91 (brd, 2H) ppm

MS: (474.3)

### EXAMPLE 10

### 3-(4-bromobenzylamino)-2-(2-chlorophenyl)quinazolin-4(3H)-one

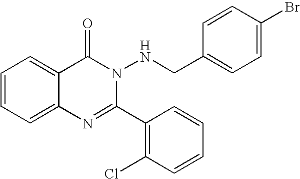

Prepared as described in Example 1.

¹H-NMR: (CDCl₃) 8.39 (d, 1H), 7.82 (dd, 2H), 7.60 (dd, 1H), 7.44 (dd, 2H), 7.26 (d, 2H), 7.22 (d, 2H), 6.72 (d, 2H), 5.74 (t, 1H), 4.04 (brd, 1H), 3.78 (brd, 1H) ppm

MS: (440.7)

### EXAMPLE 11

### 3-(4-tert-butylbenzylamino)-2-(2-fluorophenyl)quinazolin-4(3H)-one

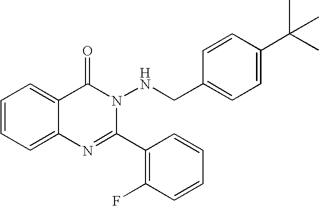

Prepared as described in Example 1.

¹H-NMR: (CDCl₃) 8.39 (d, 1H), 7.82 (dd, 2H), 7.60 (dd, 2H), 7.38 (dd, 2H), 7.22 (d, 1H), 7.18 (dd, 2H), 6.80 (d, 2H), 5.80 (t, 1H), 3.95 (brd, 2H), 1.22 (s, 9H) ppm

MS: (401.5)

### EXAMPLE 12

### 3-(2,4-difluorobenzylamino)-2-(2-fluorophenyl)quinazolin-4(3H)-one

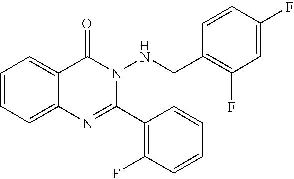

Prepared as described in Example 1.

¹H-NMR: (CDCl₃) 8.39 (d, 1H), 7.82 (dd, 2H), 7.68 (dd, 1H), 7.42 (dd, 1H), 7.20 (dd, 2H), 7.00 (s, 1H), 6.80 (dd, 1H), 6.62 (dd, 2H), 5.84 (t, 1H), 4.02 (brd, 2H) ppm

MS: (381.5)

### EXAMPLE 13

### 3-(3,5-difluorobenzylamino)-2-(2-fluorophenyl)quinazolin-4(3H)-one

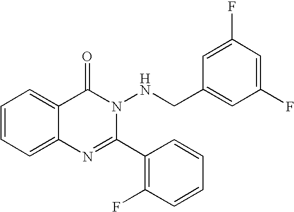

Prepared as described in Example 1.

¹H-NMR: (CDCl₃) 8.39 (d, 1H), 7.82 (dd, 2H), 7.60 (dd, 2H), 7.40 (dd, 1H), 7.22 (s, 1H), 7.20 (s, 1H), 6.63 (dd, 1H), 6.38 (d, 2H), 5.81 (t, 1H), 3.98 (brd, 2H) ppm

MS: (381.5)

### EXAMPLE 14

### 3-(3,4-difluorobenzylamino)-2-(2-fluorophenyl)quinazolin-4(3H)-one

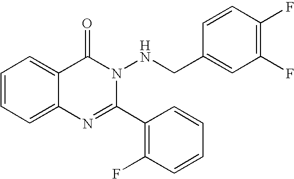

Prepared as described in Example 1.

¹H-NMR: (CDCl₃) 8.39 (d, 1H), 7.82 (dd, 2H), 7.58 (dd, 2H), 7.38 (dd, 1H), 7.20 (d, 1H), 7.18 (d, 1H), 6.96 (dd, 1H), 6.62 (dd, 1H), 6.58 (dd, 1H), 5.78 (t, 1H), 3.96 (brd, 2H) ppm

MS: (381.5)

### EXAMPLE 15

### 3-(4-bromobenzylamino)-2-o-tolylquinazolin-4(3H)-one

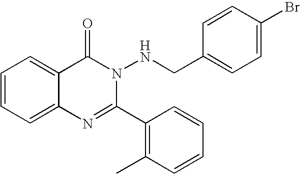

Prepared as described in Example 1.

¹H-NMR: (CDCl₃) 8.39 (d, 1H), 7.82 (d, 2H), 7.60 (dd, 1H), 7.42 (dd, 2H), 7.34 (m, 4H), 6.64 (d, 2H), 5.82 (t, 1H), 3.82 (brd, 2H), 2.24 (s, 3H) ppm

MS: (420.3)

### EXAMPLE 16

### 3-(2,4-difluorobenzylamino)-2-phenethylquinazolin-4(3H)-one

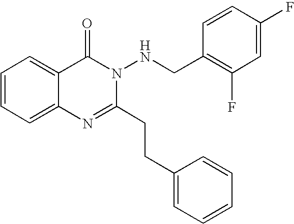

Prepared as described in Example 1.

¹H-NMR: (CDCl₃) 8.39 (d, 1H), 7.82 (dd, 1H), 7.78 (d, 1H), 7.44 (dd, 1H), 7.34 (dd, 2H), 7.30 (dd, 2H), 7.22 (dd, 2H), 6.82 (dd, 2H), 5.62 (t, 1H), 4.10 (brd, 2H), 3.20 (brt, 2H), 3.16 (brt, 2H) ppm

MS: (391.5)

### EXAMPLE 17

### 2-(2-fluorophenyl)-3-(4-(trifluoromethyl)benzylamino)quinazolin-4(3H)-one

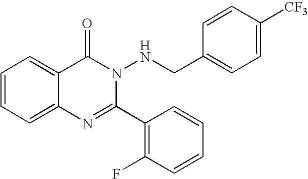

Prepared as described in Example 1.

¹H-NMR: (CDCl₃) 8.39 (d, 1H), 7.82 (dd, 2H), 7.58 (dd, 2H), 7.54 (dd, 1H), 7.40 (d, 2H), 7.20 (m, 2H), 6.98 (d, 2H), 5.82 (t, 1H), 4.02 (brd, 2H) ppm

MS: (413.4)

### EXAMPLE 18

### 3-(3-chlorobenzylamino)-2-(2-fluorophenyl)quinazolin-4(3H)-one

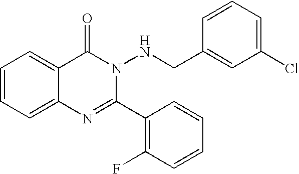

Prepared as described in Example 1.

¹H-NMR: (CDCl₃) 8.39 (d, 1H), 7.82 (dd, 2H), 7.58 (dd, 2H), 7.37 (dd, 1H), 7.22 (d, 1H), 7.20 (m, 2H), 7.10 (dd, 1H), 6.78 (d, 2H), 5.80 (t, 1H), 3.98 (brd, 2H) ppm

MS: (379.8)

### EXAMPLE 19

### 3-(4-bromobenzylamino)-2-(2-methoxyphenyl)quinazolin-4(3H)-one

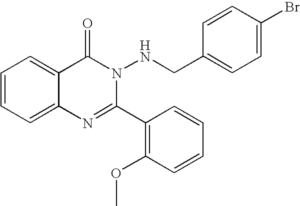

Prepared as described in Example 1.

¹H-NMR: (CDCl₃) 8.39 (d, 1H), 7.82 (d, 2H), 7.56 (dd, 2H), 7.26 (d, 2H), 7.20 (d, 1H), 7.04 (dd, 1H), 6.98 (d, 1H), 6.68 (d, 2H), 5.60 (t, 1H), 4.02 (brd, 2H), 3.78 (s, 3H) ppm

MS: (436.3)

### EXAMPLE 20

### 2-(2-fluorophenyl)-3-(3-(trifluoromethyl)benzylamino)quinazolin-4(3H)-one

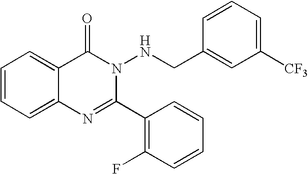

Prepared as described in Example 1.

¹H-NMR: (CDCl₃) 8.39 (d, 1H), 7.82 (dd, 2H), 7.56 (dd, 2H), 7.48 (dd, 1H), 7.40 (dd, 1H), 7.32 (m, 2H), 7.20 (m, 1H), 7.10 (d, 1H), 7.01 (s, 1H), 5.81 (t, 1H), 4.02 (brd, 2H) ppm

MS: (413.4)

### EXAMPLE 21

### 3-(3-fluoro-4-(trifluoromethyl)benzylamino)-2-(2-fluorophenyl)quinazolin-4(3H)-one

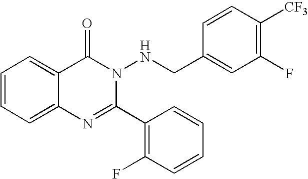

Prepared as described in Example 1.

¹H-NMR: (CDCl₃) 8.39 (d, 1H), 7.82 (dd, 2H), 7.58 (dd, 1H), 7.54 (dd, 1H), 7.40 (dd, 1H), 7.30 (dd, 1H), 7.22 (m, 2H), 6.78 (d, 1H), 6.60 (d, 1H), 5.83 (t, 1H), 4.02 (brd, 2H) ppm

MS: (431.4)

### EXAMPLE 22

### 2-(2-fluorophenyl)-3-(4-methylbenzylamino)quinazolin-4(3H)-one

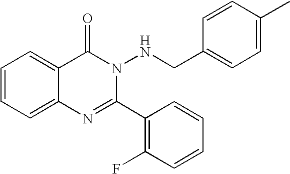

Prepared as described in Example 1.

¹H-NMR: (CDCl₃) 8.39 (d, 1H), 7.82 (dd, 2H), 7.58 (dd, 1H), 7.54 (dd, 1H), 7.40 (dd, 1H), 7.23 (d, 1H), 7.18 (dd, 2H), 6.99 (d, 1H), 6.78 (d, 2H), 5.78 (t, 1H), 3.84 (brd, 2H), 2.23 (s, 3H) ppm

MS: (359.4)

### EXAMPLE 23

### 2-(2-fluorophenyl)-3-(4-(trifluoromethoxy)benzylamino)quinazolin-4(3H)-one

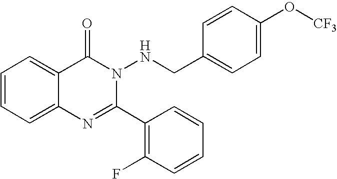

Prepared as described in Example 1.

¹H-NMR: (CDCl₃) 8.39 (d, 1H), 7.82 (dd, 2H), 7.58 (dd, 1H), 7.54 (dd, 1H), 7.40 (dd, 1H), 7.34 (dd, 1H), 7.23 (dd, 2H), 7.00 (d, 1H), 6.84 (d, 2H), 5.80 (t, 1H), 4.00 (brd, 2H) ppm

MS: (375.4)

### EXAMPLE 24

### 3-(4-bromobenzylamino)-2-(2-fluorophenyl)-7-methylquinazolin-4(3H)-one

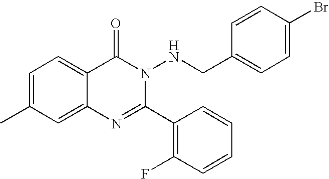

Prepared as described in Example 1.

¹H-NMR: (CDCl₃) 8.21 (d, 1H), 7.60 (s, 1H), 7.50 (dd, 2H), 7.48 (dd, 2H), 7.26 (dd, 1H), 7.18 (dd, 2H), 6.70 (d, 2H), 5.79 (t, 1H), 3.88 (brd, 2H), 2.51 (s, 3H) ppm

MS: (438.3)

### EXAMPLE 25

### 3-(2,4-difluorobenzylamino)-2-(2-fluorophenyl)-7-methylquinazolin-4(3H)-one

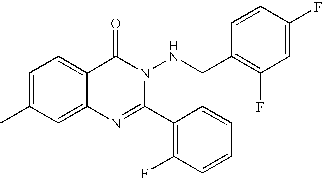

Prepared as described in Example 1.

¹H-NMR: (CDCl₃) 8.21 (d, 1H), 7.50 (s, 1H), 7.40 (dd, 1H), 7.30 (d, 1H), 7.18 (d, 1H), 7.09 (dd, 2H), 6.74 (dd, 1H), 6.59 (m, 2H), 5.79 (t, 1H), 3.96 (brd, 2H), 2.51 (s, 3H) ppm

MS: (395.4)

### EXAMPLE 26

### 3-(4-bromobenzylamino)-2-(2-fluorophenyl)-6-methoxy quinazolin-4(3H)-one

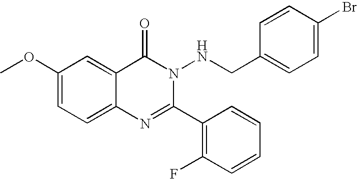

Prepared as described in Example 1.

¹H-NMR: (CDCl₃) 7.70 (d, 2H), 7.50 (dd, 1H), 7.40 (d, 1H), 7.38 (dd, 1H), 7.22 (dd, 2H), 7.18 (dd, 2H), 6.71 (d, 2H), 5.80 (t, 1H), 3.99 (s, 3H), 3.86 (brd, 2H) ppm

MS: (454.3)

### EXAMPLE 27

### 3-(2,4-difluorobenzylamino)-2-(2-fluorophenyl)-6-methoxy quinazolin-4(3H)-one

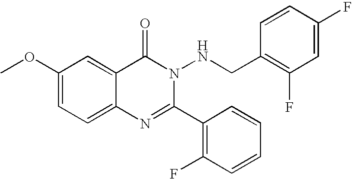

Prepared as described in Example 1.

¹H-NMR: (CDCl₃) 7.70 (d, 2H), 7.44 (dd, 1H), 7.40 (d, 1H), 7.25 (dd, 1H), 7.18 (dd, 2H), 6.80 (dd, 1H), 6.62 (dd, 2H), 5.84 (t, 1H), 3.99 (s, 3H), 3.98 (brd, 2H) ppm

MS: (411.4)

### EXAMPLE 28

### 3-(4-bromobenzylamino)-2-(2-fluorophenyl)-7-(trifluoromethyl)quinazolin-4(3H)-one

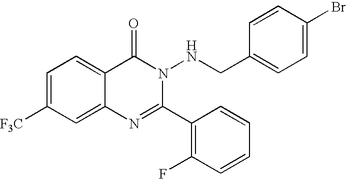

Prepared as described in Example 1.

¹H-NMR: (CDCl₃) 8.42 (d, 1H), 8.06 (s, 1H), 7.78 (d, 1H), 7.52 (dd, 1H), 7.36 (m, 2H), 7.22 (dd, 1H), 7.18 (dd, 2H), 6.70 (d, 2H), 5.81 (t, 1H), 3.90 (brd, 2H) ppm

MS: (492.3)

### EXAMPLE 29

### 3-(2,4-difluorobenzylamino)-2-(2-fluorophenyl)-7-(trifluoromethyl)quinazolin-4(3H)-one

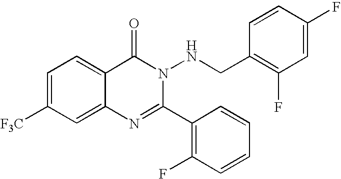

Prepared as described in Example 1.

¹H-NMR: (CDCl₃) 8.42 (d, 1H), 8.06 (s, 1H), 7.78 (d, 1H), 7.52 (dd, 1H), 7.31 (dd, 1H), 7.18 (dd, 2H), 6.80 (dd, 1H), 6.62 (dd, 2H), 5.89 (t, 1H), 4.00 (brd, 2H) ppm

MS: (449.4)

### EXAMPLE 30

### 3-(4-chlorobenzylamino)-2-(2-fluorophenyl)-7-(trifluoromethyl)quinazolin-4(3H)-one

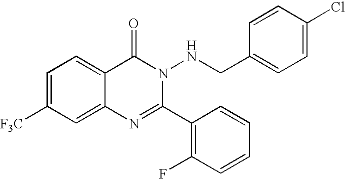

Prepared as described in Example 1.

¹H-NMR: (CDCl₃) 8.42 (d, 1H), 8.06 (s, 1H), 7.78 (d, 1H), 7.56 (dd, 1H), 7.38 (dd, 1H), 7.22 (dd, 2H), 7.18 (dd, 2H), 6.78 (d, 2H), 5.80 (t, 1H), 3.90 (brd, 2H) ppm

MS: (447.8)

### EXAMPLE 31

### 3-(4-bromobenzylamino)-2-(pyridin-2-yl)quinazolin-4(3H)-one

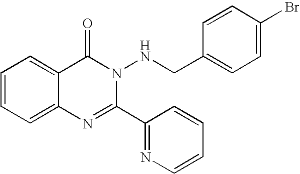

Prepared as described in Example 1.

¹H-NMR: (CDCl₃) 8.72 (d, 1H), 8.39 (d, 1H), 7.82 (dd, 2H), 7.63 (d, 1H), 7.58 (dd, 1H), 7.42 (dd, 2H), 7.26 (d, 2H), 6.86 (d, 2H), 6.04 (t, 1H), 4.11 (d, 2H) ppm

MS: (407.3)

### EXAMPLE 32

### 4-((2-(4-fluorophenyl)-4-oxoquinazolin-3(4H)-ylamino)methyl)benzonitrile

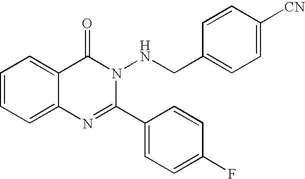

Prepared as described in Example 1.

¹H-NMR: (CDCl₃) 8.36 (d, 1H), 7.82 (dd, 2H), 7.78 (dd, 1H), 7.62 (dd, 2H), 7.50 (dd, 2H), 7.18 (dd, 2H), 7.06 (dd, 2H), 6.08 (t, 1H), 3.89 (brd, 2H) ppm

MS: (370.4)

### EXAMPLE 33

### 3-(4-bromobenzylamino)-2-(4-fluorophenyl)-7-methoxyquinazolin-4(3H)-one

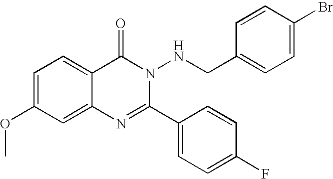

Prepared as described in Example 1.

¹H-NMR: (CDCl₃) 8.21 (d, 1H), 7.78 (dd, 2H), 7.32 (d, 2H), 7.18 (dd, 2H), 7.14 (dd, 2H), 6.81 (d, 2H), 6.00 (t, 1H), 3.92 (s, 3H), 3.79 (brd, 2H) ppm

MS: (454.3)

### EXAMPLE 34

### 3-(2,4-difluorobenzylamino)-2-(4-fluorophenyl)-7-methoxy quinazolin-4(3H)-one

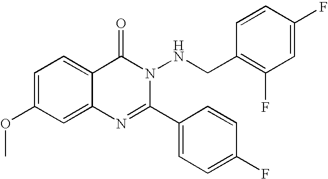

Prepared as described in Example 1.

¹H-NMR: (CDCl₃) 8.21 (d, 1H), 7.78 (dd, 2H), 7.12 (dd, 2H), 7.08 (dd, 2H), 6.88 (dd, 1H), 6.62 (m, 2H), 6.12 (t, 1H), 3.92 (s, 3H), 3.84 (brd, 2H) ppm

MS: (411.4)

### EXAMPLE 35

### 3-(4-chlorobenzylamino)-2-(4-fluorophenyl)-7-methoxyquinazolin-4(3H)-one

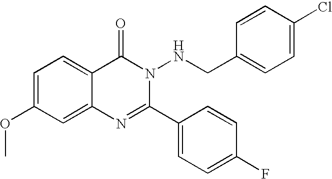

Prepared as described in Example 1.

¹H-NMR: (CDCl₃) 8.21 (d, 1H), 7.78 (dd, 2H), 7.18 (dd, 2H), 7.14 (dd, 2H), 7.10 (dd, 2H), 6.88 (dd, 2H), 6.00 (t, 1H), 3.92 (s, 3H), 3.79 (brd, 2H) ppm

MS: (409.8)

### EXAMPLE 36

### 2-(4-fluorophenyl)-7-methoxy-3-(4-(trifluoromethyl)benzylamino)quinazolin-4(3H)-one

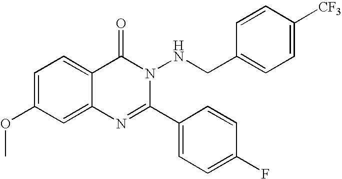

Prepared as described in Example 1.

¹H-NMR: (CDCl₃) 8.21 (d, 1H), 7.76 (dd, 2H), 7.42 (d, 2H), 7.14 (d, 2H), 7.10 (dd, 2H), 7.06 (d, 2H), 6.04 (t, 1H), 3.92 (s, 3H), 3.79 (brd, 2H) ppm

MS: (443.4)

### EXAMPLE 37

### 2-(4-fluorophenyl)-3-(pyridin-3-ylmethylamino)quinazolin-4(3H)-one

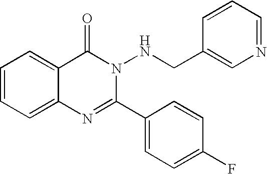

Prepared as described in Example 1.

¹H-NMR: (CDCl₃) 8.49 (d, 1H), 8.32 (d, 1H), 8.21 (s, 1H), 7.82 (dd, 2H), 7.78 (dd, 2H), 7.58 (dd, 1H), 7.27 (dd, 1H), 7.18 (dd, 2H), 7.14 (d, 1H), 6.06 (t, 1H), 3.82 (brd, 2H) ppm

MS: (346.4)

### EXAMPLE 38

### 2-(4-fluorophenyl)-3-(naphthalen-2-ylmethylamino)quinazolin-4(3H)-one

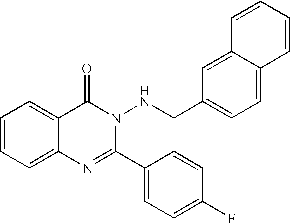

Prepared as described in Example 1.

¹H-NMR: (CDCl₃) 8.38 (d, 1H), 7.82 (dd, 2H), 7.81 (d, 2H), 7.72 (dd, 2H), 7.58 (dd, 2H), 7.54 (dd, 2H), 7.11 (dd, 2H), 7.08 (dd, 2H), 6.16 (t, 1H), 4.02 (brd, 2H) ppm

MS: (395.4)

### EXAMPLE 39

### 2-(4-fluorophenyl)-3-(4-morpholinobutylamino)quinazolin-4(3H)-one

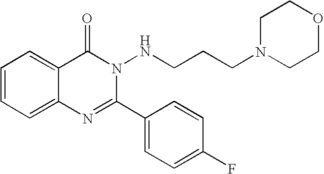

Prepared as described in Example 1.

¹H-NMR: (CDCl₃) 8.30 (d, 1H), 7.92 (dd, 2H), 7.79 (d, 2H), 7.50 (dd, 1H), 7.16 (dd, 2H), 6.44 (t, 1H), 3.72 (m, 2H), 3.68 (t, 2H), 2.42 (m, 2H), 2.36 (m, 4H), 2.22 (m, 2H), 1.70 (m, 2H) ppm

MS: (396.5)

### EXAMPLE 40

### 3-(4-bromobenzylamino)-2-(furan-2-yl)quinazolin-4(3H)-one

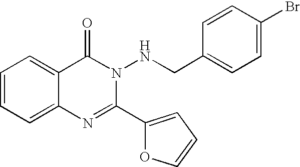

Prepared as described in Example 1.

¹H-NMR: (CDCl₃) 8.28 (d, 1H), 7.88 (d, 1H), 7.85 (dd, 2H), 7.78 (d, 1H), 7.52 (d, 2H), 7.50 (d, 1H), 7.31 (d, 2H), 6.62 (d, 1H), 5.92 (t, 1H), 4.02 (brd, 2H) ppm

MS: (396.2)

### EXAMPLE 41

### 3-(bis(4-bromobenzyl)amino)-2-(furan-2-yl)quinazolin-4(3H)-one

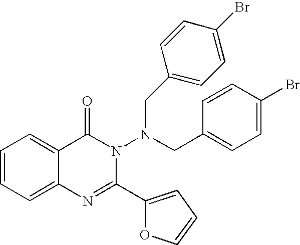

Prepared as described in Example 1.

¹H-NMR: (CDCl₃) 8.30 (d, 1H), 7.78 (d, 1H), 7.64 (d, 1H), 7.58 (d, 1H), 7.50 (dd, 1H), 7.26 (d, 2H), 7.24 (d, 2H), 7.22 (d, 1H), 7.02 (d, 2H), 6.99 (d, 2H), 6.50 (d, 1H), 4.70 (d, 2H), 4.47 (d, 2H) ppm

MS: (565.3)

### EXAMPLE 42

### 3-(4-bromobenzylamino)-2-(4-(dimethylamino)phenyl)quinazolin-4(3H)-one

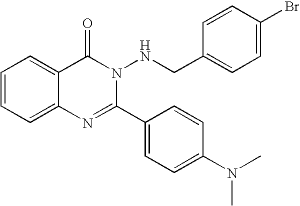

Prepared as described in Example 1.

¹H-NMR: (CDCl₃) 8.26 (d, 1H), 7.90 (d, 2H), 7.78 (d, 2H), 7.43 (dd, 1H), 7.36 (d, 2H), 6.99 (d, 2H), 6.73 (d, 2H), 6.18 (t, 1H), 3.79 (brd, 2H), 3.04 (s, 6H) ppm

MS: (449.3)

### EXAMPLE 43

### 3-(4-chlorobenzylamino)-2-(4-(dimethylamino)phenyl)quinazolin-4(3H)-one

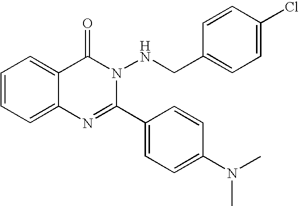

Prepared as described in Example 1.

¹H-NMR: (CDCl₃) 8.26 (d, 1H), 7.90 (d, 2H), 7.78 (d, 2H), 7.43 (dd, 1H), 7.20 (d, 2H), 7.05 (d, 2H), 6.78 (d, 2H), 6.18 (t, 1H), 3.79 (brd, 2H), 3.04 (s, 6H) ppm

MS: (404.9)

### EXAMPLE 44

### 3-(4-bromobenzylamino)-2-cyclohexylquinazolin-4(3H)-one

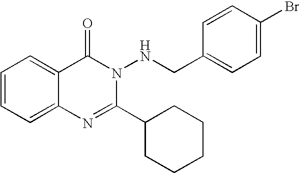

Prepared as described in Example 1.

¹H-NMR: (CDCl₃) 8.22 (d, 1H), 7.72 (dd, 1H), 7.68 (d, 1H), 7.51 (d, 2H), 7.42 (dd, 1H), 7.26 (d, 2H), 5.62 (t, 1H), 4.02 (brd, 2H), 3.22 (m, 1H), 1.90 (m, 4H), 1.78 (m, 2H), 1.38 (m, 4H) ppm

MS: (412.3)

### EXAMPLE 45

### 3-(4-chlorobenzylamino)-2-cyclohexylquinazolin-4(3H)-one

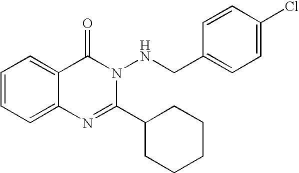

Prepared as described in Example 1.

¹H-NMR: (CDCl₃) 8.24 (d, 1H), 7.78 (dd, 1H), 7.68 (d, 1H), 7.42 (dd, 1H), 7.26 (m, 4H), 5.62 (t, 1H), 4.02 (brd, 2H), 3.24 (m, 1H), 1.90 (m, 4H), 1.78 (m, 2H), 1.38 (m, 4H) ppm

MS: (367.9)

### EXAMPLE 46

### 3-(4-bromobenzylamino)-2-(3-chlorophenyl)quinazolin-4(3H)-one

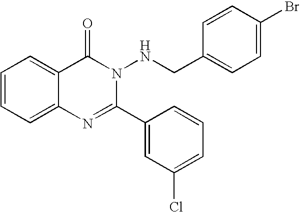

Prepared as described in Example 1.

¹H-NMR: (CDCl₃) 8.34 (d, 1H), 7.81 (dd, 2H), 7.78 (s, 1H), 7.62 (d, 1H), 7.58 (dd, 1H), 7.48 (d, 1H), 7.40 (dd, 1H), 7.31 (d, 2H), 6.81 (d, 2H), 6.01 (t, 1H), 3.82 (brd, 2H) ppm

MS: (440.7)

### EXAMPLE 47

### 3-(4-bromobenzylamino)-2-(4-chlorophenyl)quinazolin-4(3H)-one

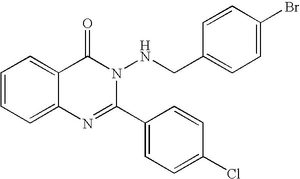

Prepared as described in Example 1.

¹H-NMR: (CDCl₃) 8.34 (d, 1H), 7.81 (dd, 2H), 7.78 (d, 2H), 7.58 (dd, 1H), 7.42 (d, 2H), 7.32 (d, 2H), 6.82 (d, 2H), 6.02 (t, 1H), 3.80 (brd, 2H) ppm

MS: (440.7)

### EXAMPLE 48

### 3-(4-chlorobenzylamino)-2-(3-chlorophenyl)quinazolin-4(3H)-one

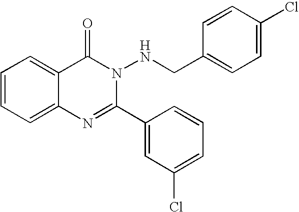

Prepared as described in Example 1.

¹H-NMR: (CDCl₃) 8.34 (d, 1H), 7.80 (dd, 2H), 7.74 (s, 1H), 7.62 (d, 1H), 7.56 (dd, 1H), 7.48 (dd, 1H), 7.40 (dd, 1H), 7.19 (d, 2H), 6.89 (d, 2H), 6.02 (t, 1H), 3.82 (brd, 2H) ppm

MS: (396.3)

### EXAMPLE 49

### 2-(3-chlorophenyl)-3-(4-(trifluoromethyl)benzylamino)quinazolin-4(3H)-one

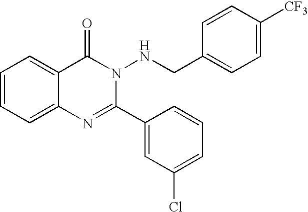

Prepared as described in Example 1.

¹H-NMR: (CDCl₃) 8.34 (d, 1H), 7.80 (dd, 2H), 7.79 (s, 1H), 7.62 (dd, 1H), 7.58 (dd, 1H), 7.46 (dd, 1H), 7.44 (d, 2H), 7.38 (dd, 1H), 7.08 (d, 2H), 6.04 (t, 1H), 3.92 (brd, 2H) ppm

MS: (429.8)

### EXAMPLE 50

### 2-(3-chlorophenyl)-3-(3,4-difluorobenzylamino)quinazolin-4(3H)-one

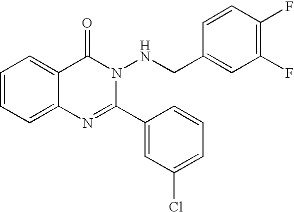

Prepared as described in Example 1.

¹H-NMR: (CDCl₃) 8.34 (d, 1H), 7.80 (dd, 1H), 7.79 (s, 1H), 7.62 (d, 1H), 7.58 (dd, 1H), 7.46 (dd, 1H), 7.40 (dd, 2H), 6.99 (dd, 1H), 6.71 (m, 2H), 6.01 (t, 1H), 3.81 (brd, 2H) ppm

MS: (397.8)

### EXAMPLE 51

### 3-(4-chlorobenzylamino)-2-(4-chlorophenyl)quinazolin-4(3H)-one

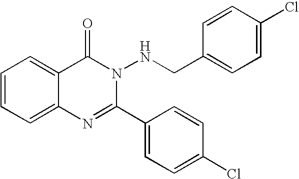

Prepared as described in Example 1.

¹H-NMR: (CDCl₃) 8.34 (d, 1H), 7.80 (dd, 2H), 7.79 (dd, 2H), 7.58 (dd, 1H), 7.42 (d, 2H), 7.18 (d, 2H), 6.90 (d, 2H), 6.02 (t, 1H), 3.80 (brd, 2H) ppm

MS: (396.2)

### EXAMPLE 52

### 2-(4-chlorophenyl)-3-(4-(trifluoromethyl)benzylamino)quinazolin-4(3H)-one

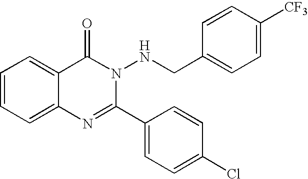

Prepared as described in Example 1.

¹H-NMR: (CDCl₃) 8.34 (d, 1H), 7.80 (dd, 2H), 7.70 (d, 2H), 7.58 (dd, 1H), 7.43 (d, 2H), 7.40 (d, 2H), 7.06 (d, 2H), 6.09 (t, 1H), 3.90 (brd, 2H) ppm

MS: (429.8)

### EXAMPLE 53

### 2-(4-chlorophenyl)-3-(3,4-difluorobenzylamino)quinazolin-4(3H)-one

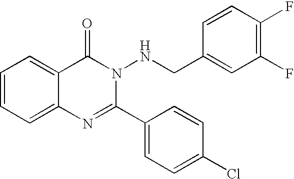

Prepared as described in Example 1.

¹H-NMR: (CDCl₃) 8.34 (d, 1H), 7.80 (dd, 2H), 7.78 (d, 1H), 7.58 (dd, 1H), 7.43 (d, 2H), 7.00 (dd, 2H), 6.72 (m, 2H), 6.04 (t, 1H), 3.80 (brd, 2H) ppm

MS: (397.8)

### EXAMPLE 54

### 3-(4-bromobenzylamino)-2-(4-fluoro-3-methylphenyl)quinazolin-4(3H)-one

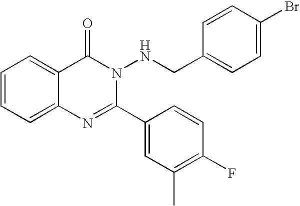

Prepared as described in Example 1.

¹H-NMR: (CDCl₃) 8.34 (d, 1H), 7.80 (dd, 2H), 7.62 (dd, 1H), 7.56 (dd, 2H), 7.32 (d, 2H), 7.04 (dd, 1H), 6.62 (d, 2H), 6.02 (t, 1H), 3.80 (brd, 2H), 2.34 (s, 3H) ppm

MS: (438.3)

### EXAMPLE 55

### 3-(4-bromobenzylamino)-2-(3-methoxyphenyl)quinazolin-4(3H)-one

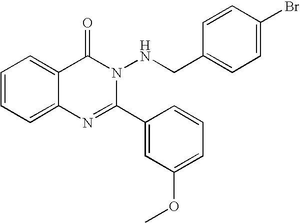

Prepared as described in Example 1.

¹H-NMR: (CDCl₃) 8.34 (d, 1H), 7.80 (dd, 2H), 7.52 (dd, 1H), 7.40 (d, 2H), 7.30 (d, 2H), 7.26 (s, 1H), 7.04 (m, 1H), 6.82 (d, 2H), 6.01 (t, 1H), 3.84 (s, 3H), 3.80 (brd, 2H) ppm

MS: (436.3)

### EXAMPLE 56

### 3-(4-chlorobenzylamino)-2-(3-methoxyphenyl)quinazolin-4(3H)-one

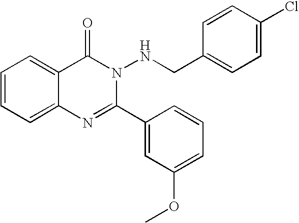

Prepared as described in Example 1.

¹H-NMR: (CDCl₃) 8.34 (d, 1H), 7.80 (d, 2H), 7.58 (dd, 1H), 7.40 (d, 2H), 7.26 (s, 1H), 7.18 (d, 2H), 7.05 (dd, 1H), 6.90 (d, 2H), 6.01 (t, 1H), 3.84 (s, 3H), 3.80 (brd, 2H) ppm

MS: (391.8)

### EXAMPLE 57

### 3-(4-chlorobenzylamino)-2-(4-fluoro-3-methylphenyl)quinazolin-4(3H)-one

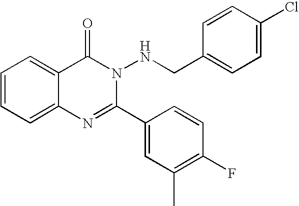

Prepared as described in Example 1.

¹H-NMR: (CDCl₃) 8.34 (d, 1H), 7.80 (dd, 2H), 7.64 (dd, 1H), 7.56 (dd, 2H), 7.21 (s, 1H), 7.18 (d, 1H), 7.08 (dd, 1H), 6.90 (d, 2H), 6.03 (t, 1H), 3.80 (brd, 2H), 2.36 (s, 3H) ppm

MS: (393.8)

### EXAMPLE 58

### 3-(4-bromobenzylamino)-2-(3,5-dimethoxyphenyl)quinazolin-4(3H)-one

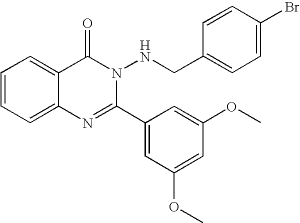

Prepared as described in Example 1.

¹H-NMR: (CDCl₃) 8.34 (d, 1H), 7.80 (d, 2H), 7.56 (dd, 1H), 7.21 (s, 1H), 7.19 (d, 1H), 6.92 (d, 2H), 6.90 (s, 2H), 6.60 (s, 1H), 6.00 (t, 1H), 3.82 (brd, 2H), 3.80 (s, 6H) ppm

MS: (466.3)

### EXAMPLE 59

### 3-(4-chlorobenzylamino)-2-(3,5-dimethoxyphenyl)quinazolin-4(3H)-one

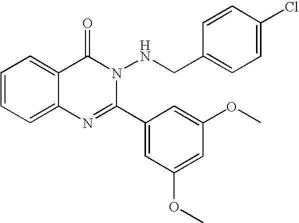

Prepared as described in Example 1.

¹H-NMR: (CDCl₃) 8.34 (d, 1H), 7.80 (d, 2H), 7.56 (dd, 1H), 7.21 (s, 1H), 7.19 (d, 1H), 6.92 (d, 2H), 6.90 (s, 2H), 6.60 (s, 1H), 6.00 (t, 1H), 3.82 (brd, 2H), 3.80 (s, 6H) ppm

MS: (421.9)

### EXAMPLE 60

### 3-(4-bromobenzylamino)-2-(3-(trifluoromethoxy)phenyl)quinazolin-4(3H)-one

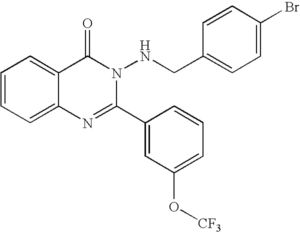

Prepared as described in Example 1.

¹H-NMR: (CDCl₃) 8.34 (d, 1H), 7.80 (dd, 2H), 7.70 (dd, 2H), 7.58 (dd, 1H), 7.48 (dd, 1H), 7.39 (d, 1H), 7.25 (d, 2H), 6.80 (d, 2H), 6.00 (t, 1H), 3.80 (brd, 2H) ppm

MS: (490.3)

### EXAMPLE 61

### 3-(4-chlorobenzylamino)-2-(3-(trifluoromethoxy)phenyl)quinazolin-4(3H)-one

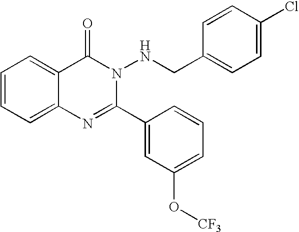

Prepared as described in Example 1.

¹H-NMR: (CDCl₃) 8.34 (d, 1H), 7.80 (dd, 2H), 7.70 (dd, 2H), 7.58 (dd, 1H), 7.48 (dd, 1H), 7.39 (d, 1H), 7.19 (d, 2H), 6.84 (d, 2H), 6.00 (t, 1H), 3.80 (brd, 2H) ppm

MS: (445.8)

### EXAMPLE 62

### 3-(4-chlorobenzylamino)-2-(3,4-dimethoxyphenyl)quinazolin-4(3H)-one

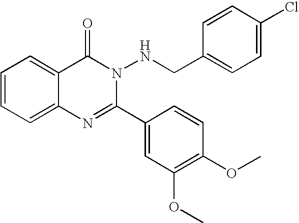

Prepared as described in Example 1.

¹H-NMR: (CDCl₃) 8.34 (d, 1H), 7.80 (d, 2H), 7.52 (dd, 2H), 7.42 (s, 1H), 7.19 (d, 2H), 6.99 (d, 2H), 6.97 (d, 1H), 6.13 (t, 1H), 4.00 (s, 3H), 3.92 (s, 3H), 3.80 (brd, 2H) ppm

MS: (421.9)

### EXAMPLE 63

### 2-(benzo[d][1,3]dioxol-5-yl)-3-(4-bromobenzylamino)quinazolin-4(3H)-one

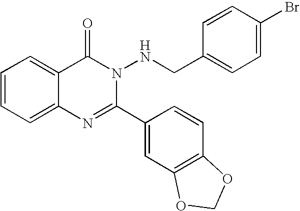

Prepared as described in Example 1.

¹H-NMR: (CDCl₃) 8.34 (d, 1H), 7.80 (dd, 2H), 7.52 (dd, 1H), 7.42 (d, 1H), 7.37 (s, 1H), 7.30 (d, 2H), 6.92 (d, 2H), 6.70 (d, 1H), 6.10 (t, 1H), 6.05 (s, 2H), 3.80 (brd, 2H) ppm

MS: (450.3)

### EXAMPLE 64

### 2-(benzo[d][1,3]dioxol-5-yl)-3-(4-chlorobenzylamino)quinazolin-4(3H)-one

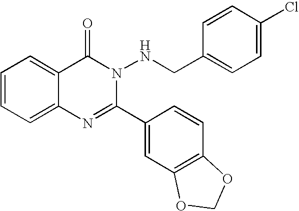

Prepared as described in Example 1.

¹H-NMR: (CDCl₃) 8.34 (d, 1H), 7.80 (dd, 2H), 7.52 (dd, 1H), 7.42 (d, 1H), 7.37 (s, 1H), 7.20 (d, 2H), 7.02 (d, 2H), 6.89 (d, 1H), 6.10 (t, 1H), 6.05 (s, 2H), 3.80 (brd, 2H) ppm

MS: (405.8)

### EXAMPLE 65

### 3-(4-bromobenzylamino)-2-(3-methoxy-4-methylphenyl)quinazolin-4(3H)-one

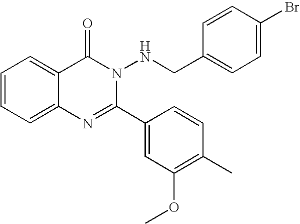

Prepared as described in Example 1.

¹H-NMR: (CDCl₃) 8.34 (d, 1H), 7.80 (dd, 2H), 7.54 (dd, 1H), 7.39 (d, 1H), 7.30 (s, 1H), 7.24 (dd, 1H), 7.18 (d, 2H), 6.92 (d, 2H), 6.04 (t, 1H), 3.82 (s, 3H), 3.80 (brd, 2H), 2.30 (s, 3H) ppm

MS: (450.3)

### EXAMPLE 66

### 3-(4-chlorobenzylamino)-2-(3-methoxy-4-methylphenyl)quinazolin-4(3H)-one

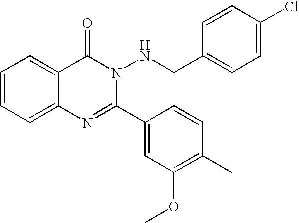

Prepared as described in Example 1.

¹H-NMR: (CDCl₃) 8.34 (d, 1H), 7.80 (d, 2H), 7.54 (dd, 1H), 7.39 (d, 1H), 7.24 (d, 1H), 7.21 (s, 1H), 7.00 (dd, 2H), 6.90 (dd, 2H), 6.04 (t, 1H), 3.82 (s, 3H), 3.80 (brd, 2H), 2.32 (s, 3H) ppm

MS: (405.9)

### EXAMPLE 67

### 3-(4-fluorobenzylamino)-2-(3-methoxy-4-methylphenyl)quinazolin-4(3H)-one

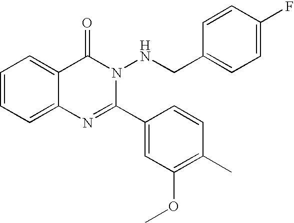

Prepared as described in Example 1.

¹H-NMR: (CDCl₃) 8.34 (d, 1H), 7.80 (d, 2H), 7.54 (dd, 1H), 7.43 (d, 1H), 7.30 (s, 1H), 7.21 (d, 1H), 7.14 (d, 2H), 6.92 (d, 2H), 6.10 (t, 1H), 3.90 (brd, 2H), 3.83 (s, 3H), 2.31 (s, 3H) ppm

MS: (389.4)

### EXAMPLE 68

### 2-(3-methoxy-4-methylphenyl)-3-(4-(trifluoromethyl)benzylamino)quinazolin-4(3H)-one

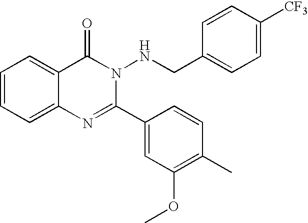

Prepared as described in Example 1.

¹H-NMR: (CDCl₃) 8.34 (d, 1H), 7.80 (d, 2H), 7.54 (dd, 1H), 7.43 (d, 2H), 7.30 (s, 1H), 7.21 (d, 1H), 7.14 (d, 1H), 6.92 (d, 2H), 6.10 (t, 1H), 3.90 (brd, 2H), 3.83 (s, 3H), 2.31 (s, 3H) ppm

MS: (439.4)

### EXAMPLE 69

### 3-(3,4-difluorobenzylamino)-2-(3-methoxy-4-methyl phenyl)quinazolin-4(3H)-one

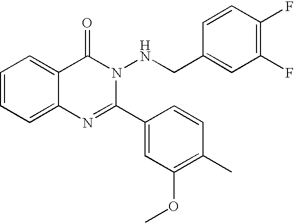

Prepared as described in Example 1.

¹H-NMR: (CDCl₃) 8.34 (d, 1H), 7.80 (d, 2H), 7.54 (dd, 1H), 7.38 (d, 1H), 7.21 (s, 1H), 6.99 (dd, 2H), 6.78 (dd, 2H), 6.04 (t, 1H), 3.82 (s, 3H), 3.80 (brd, 2H), 2.31 (s, 3H) ppm

MS: (450.3)

### EXAMPLE 70

### 3-(4-bromobenzylamino)-2-(4-fluorophenyl)-8-methylquinazolin-4(3H)-one

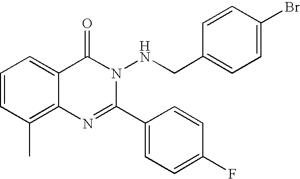

Prepared as described in Example 1.

¹H-NMR: (CDCl₃) 8.34 (d, 1H), 7.88 (dd, 2H), 7.62 (d, 1H), 7.42 (dd, 1H), 7.32 (d, 2H), 7.12 (dd, 2H), 6.83 (d, 2H), 6.04 (t, 1H), 3.80 (brd, 2H), 2.61 (s, 3H) ppm

MS: (438.3)

### EXAMPLE 71

### 3-(4-chlorobenzylamino)-2-(4-fluorophenyl)-8-methylquinazolin-4(3H)-one

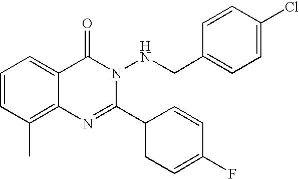

Prepared as described in Example 1.

¹H-NMR: (CDCl₃) 8.34 (d, 1H), 7.90 (dd, 2H), 7.62 (d, 1H), 7.42 (dd, 1H), 7.18 (d, 2H), 7.14 (d, 2H), 6.91 (d, 2H), 6.04 (t, 1H), 3.80 (brd, 2H), 2.61 (s, 3H) ppm

MS: (393.8)

### EXAMPLE 72

### 3-(4-bromobenzylamino)-2-(4-fluoro-3-methoxyphenyl)quinazolin-4(3H)-one

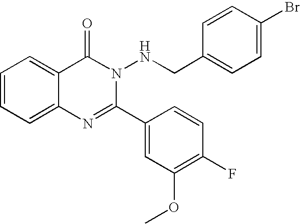

Prepared as described in Example 1.

¹H-NMR: (CDCl₃) 8.34 (d, 1H), 7.80 (dd, 2H), 7.58 (dd, 1H), 7.42 (dd, 1H), 7.38 (s, 1H), 7.31 (d, 2H), 7.18 (dd, 1H), 6.82 (d, 2H), 6.04 (t, 1H), 3.91 (s, 3H), 3.80 (brd, 2H) ppm

MS: (454.3)

### EXAMPLE 73

### 3-(4-chlorobenzylamino)-2-(4-fluoro-3-methoxyphenyl)quinazolin-4(3H)-one

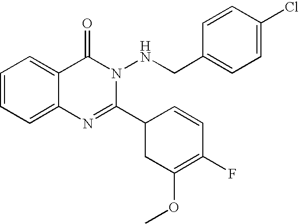

Prepared as described in Example 1.

¹H-NMR: (CDCl₃) 8.34 (d, 1H), 7.80 (dd, 2H), 7.58 (dd, 1H), 7.42 (dd, 1H), 7.38 (dd, 1H), 7.18 (d, 2H), 7.14 (s, 1H), 6.92 (d, 2H), 6.04 (t, 1H), 3.91 (s, 3H), 3.80 (brd, 2H) ppm

MS: (409.8)

### EXAMPLE 74

### 3-(4-Bromo-benzylamino)-2-(4-chloro-3-methoxy-phenyl)-3H-quinazolin-4-one

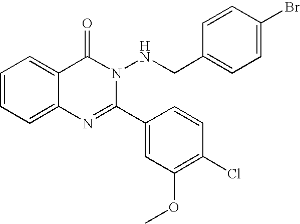

Prepared as described in Example 1.

¹H-NMR: (CDCl₃) 8.34 (d, 1H), 7.80 (dd, 2H), 7.56 (dd, 1H), 7.42 (d, 1H), 7.39 (d, 1H), 7.30 (dd, 2H), 7.21 (s, 1H), 6.82 (d, 2H), 6.04 (t, 1H), 3.91 (s, 3H), 3.80 (brd, 2H) ppm

MS: (470.8)

### EXAMPLE 75

### 3-(4-Chloro-benzylamino)-2-(4-chloro-3-methoxy-phenyl)-3H-quinazolin-4-one

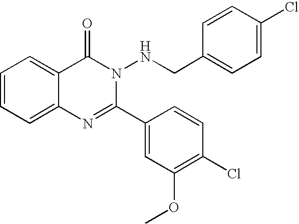

Prepared as described in Example 1.

¹H-NMR: (CDCl₃) 8.34 (d, 1H), 7.80 (dd, 2H), 7.56 (dd, 1H), 7.42 (d, 1H), 7.39 (d, 1H), 7.30 (s, 1H), 7.19 (d, 2H), 6.90 (d, 2H), 6.02 (t, 1H), 3.92 (s, 3H), 3.81 (brd, 2H) ppm

MS: (426.3)

### EXAMPLE 76

### 3-(4-Bromo-benzylamino)-2-(4-fluoro-phenyl)-8-methoxy-3H-quinazolin-4-one

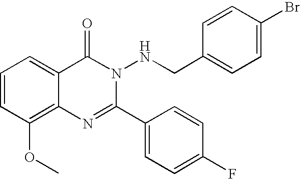

Prepared as described in Example 1.

¹H-NMR: (CDCl₃) 7.90 (d, 1H), 7.81 (dd, 2H), 7.48 (dd, 1H), 7.32 (d, 2H), 7.21 (d, 1H), 7.11 (dd, 2H), 6.82 (d, 2H), 6.04 (t, 1H), 4.01 (s, 3H), 3.78 (brd, 2H) ppm

MS: (454.3)

### EXAMPLE 77

### 3-(4-Chloro-benzylamino)-2-(4-fluoro-phenyl)-8-methoxy-3H-quinazolin-4-one

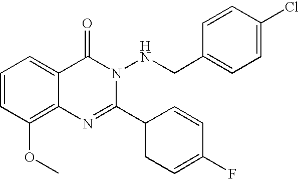

Prepared as described in Example 1.

¹H-NMR: (CDCl₃) 7.90 (d, 1H), 7.81 (dd, 2H), 7.48 (dd, 1H), 7.22 (d, 1H), 7.16 (dd, 2H), 7.15 (dd, 2H), 6.89 (d, 2H), 6.04 (t, 1H), 4.01 (s, 3H), 3.78 (brd, 2H) ppm

MS: (409.8)

### EXAMPLE 78

### 2-(4-fluorophenyl)-3-(4-(trifluoromethyl)benzylamino)quinazolin-4(3H)-one

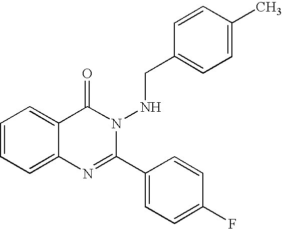

Prepared as described in Example 1.

¹H-NMR (400 MHz, DMSO-d₆): δ 8.22 (dd, 1H), 7.85 (t, 1H), 7.68 (d, 1H), 7.60 (m, 3H), 7.45 (d, 2H), 7.19 (t, 2H), 7.05 (d, 1H), 6.88 (t, 1H), 4.02 (bs, 2H).

MS: 414.21 (M+1).

### EXAMPLE 79

### 3-(4-chlorobenzylamino)-2-(4-fluorophenyl)quinazolin-4(3H)-one

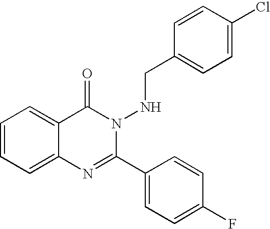

Prepared as described in Example 1.

¹H-NMR (400 MHz, CDCl₃): δ 8.32 (dd, 1H), 7.81 (m, 4H), 7.54 (t, 1H), 7.17 (m, 4H), 6.90 (d, 2H), 6.04 (t, 1H), 3.79 (bs, 2H).

MS: 379.98 (M+1).

### EXAMPLE 80

### 3-(2,4-difluorobenzylamino)-2-(4-fluorophenyl)quinazolin-4(3H)-one

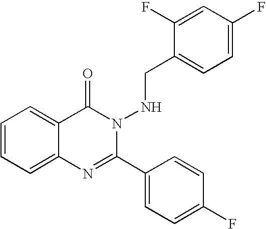

Prepared as described in Example 1.

¹H-NMR (400 MHz, CDCl₃): δ 8.30 (dd, 1H), 7.77 (m, 4H), 7.54 (t, 1H), 7.10 (t, 2H), 6.89 (t, 2H), 6.65 (m, 2H), 6.15 (t, 1H), 3.88 (bs, 2H).

MS: 382.53 (M+1).

### EXAMPLE 81

### 3-(4-bromobenzylamino)-2-(4-fluorophenyl)-7-methylquinazolin-4(3H)-one

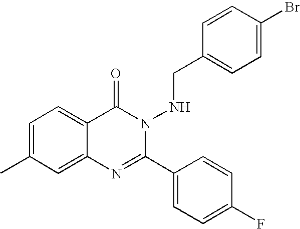

Prepared as described in Example 1.

¹H-NMR (400 MHz, CDCl₃): δ 8.20 (d, 1H), 7.79 (q, 2H), 7.56 (s, 1H), 7.37 (d, 1H), 7.31 (d, 2H), 7.14 (t, 2H), 6.83 (d, 2H), 6.03 (t, 1H), 3.76 (bs, 2H), 2.53 (s, 3H).

MS: 439.03 (M+1).

### EXAMPLE 82

### 2-(4-fluorophenyl)-7-methyl-3-(4 (trifluoromethyl)benzylamino)quinazolin-4(3H)-one

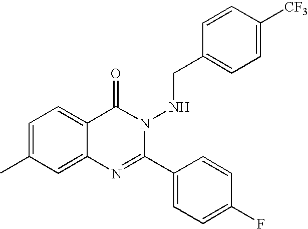

Prepared as described in Example 1.

¹H-NMR (400 MHz, CDCl₃): δ 8.21 (d, 1H), 7.75 (q, 2H), 7.56 (s, 1H), 7.44 (d, 2H), 7.36 (dd, 1H), 7.11 (t, 2H), 7.06 (d, 2H), 6.08 (t, 1H), 3.89 (bs, 2H), 2.53 (s, 3H).

MS: 428.27 (M+1).

### EXAMPLE 83

### 3-(4-bromobenzylamino)-2-(4-fluorophenyl)-6,7-dimethoxy-quinazolin-4(3H)-one

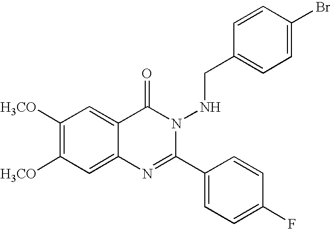

Prepared as described in Example 1.

¹H-NMR (400 MHz, CDCl₃): δ 7.78 (q, 2H), 7.62 (s, 1H), 7.32 (d, 2H), 7.16 (s, 1H), 7.13 (t, 2H), 6.84 (d, 2H), 6.06 (t, 1H), 4.04 (s, 3H), 4.01 (s, 3H), 3.76 (bs, 2H).

MS: 484.07 (M+1).

### EXAMPLE 84

### 3-(3-bromophenethylamino)-2-(4-fluorophenyl)quinazolin-4(3H)-one

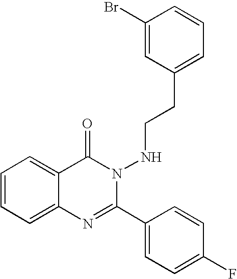

Prepared as described in Example 1 with 1-bromo-3-(2-bromoethyl)benzene was used in place of 1-bromo-4-(bromomethyl)benzene.

¹H-NMR (400 MHz, CDCl₃): δ 8.28 (d, 1H), 7.84 (q, 3H), 7.76 (m, 2H), 7.53 (m, 2H), 7.30 (d, 1H), 7.15 (s, 1H), 7.07 (t, 3H), 6.92 (d, 1H), 2.92 (bs, 2H), 2.61 (t, 2H).

MS: 439.05 (M+1).

### EXAMPLE 85

### 3-(4-bromophenethylamino)-2-(4-fluorophenyl)quinazolin-4(3H)-one

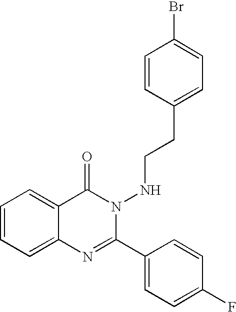

Prepared as described in Example 1 with 1-bromo-4-(2-bromoethyl)benzene was used in place of 1-bromo-4-(bromomethyl)benzene.

¹H-NMR (400 MHz, CDCl₃): δ 8.28 (d, 1H), 7.84 (q, 3H), 7.76 (m, 2H), 7.53 (m, 2H), 7.30 (d, 1H), 7.15 (s, 1H), 7.07 (t, 3H), 6.92 (d, 1H), 2.92 (bs, 2H), 2.61 (t, 2H).

MS: 439.05 (M+1).

### EXAMPLE 86

### 3-(4-bromobenzylamino)-2-(thiophen-2-yl)quinazolin-4(3H)-one

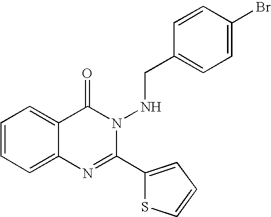

Prepared as described in Example 1.

¹H-NMR (400 MHz, CDCl₃): δ 8.44 (dd, 1H), 8.28 (dd, 1H), 7.84 (q, 3H), 7.76 (m, 2H), 7.61 (d, 1H), 7.51 (m, 3H), 7.37 (d, 1H), 7.19 (t, 2H), 5.84 (t, 1H), 4.06 (bs, 2H).

MS: 413.00 (M+1).

### EXAMPLE 87

### 2-(thiophen-2-yl)-3-(4-(trifluoromethyl)benzylamino)quinazolin-4(3H)-one

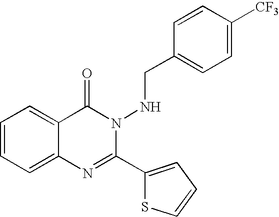

Prepared as described in Example 1.

¹H-NMR (400 MHz, CDCl₃): δ 8.42 (dd, 1H), 8.27 (d, 1H), 7.76 (m, 2H), 7.61 (m, 5H), 7.47 (sep, 1H), 7.36 (d, 1H), 7.18 (t, 1H), 5.91 (t, 1H), 4.15 (bs, 2H).

MS: 402.1 (M+1).

### EXAMPLE 88

### 3-(bis(4-(trifluoromethyl)benzyl)amino)-2-(thiophen-2-yl)quinazolin-4(3H)-one

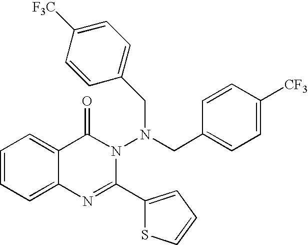

Prepared as described in Example 1.

¹H-NMR (400 MHz, CDCl₃): δ 8.21 (dd, 1H), 7.61 (m, 2H), 7.53 (m, 2H), 7.41 (m, 1H), 7.30 (q, 7H), 6.94 (t, 1H), 4.85 (d, 2H), 4.54 (d, 2H).

MS: 559.94 (M+1).

### EXAMPLE 89

### 3-(4-bromobenzylamino)-2-(thiophen-3-yl)quinazolin-4(3H)-one

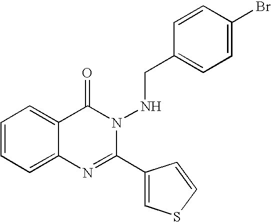

Prepared as described in Example 1.

¹H-NMR (400 MHz, CDCl₃): δ 8.35 (dd, 1H), 8.28 (dd, 1H), 7.82 (d, 1H), 7.76 (m, 2H), 7.51 (sep, 1H), 7.41 (d, 2H), 7.37 (t, 1H), 7.07 (d, 2H), 6.09 (t, 1H), 3.86 (bs, 2H).

MS: 413.02 (M+1).

### EXAMPLE 90

### 3-((4-bromobenzyl)(methyl)amino)-2-(4-fluorophenyl)quinazolin-4(3H)-one

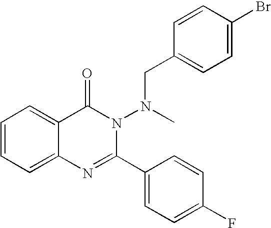

Prepared as described in Example 1 using methylhydrazine.

¹H-NMR (400 MHz, DMSO-d₆): δ 8.20 (d, 1H), 7.84 (t, 1H), 7.65 (d, 1H), 7.57 (t, 1H), 7.50 (q, 2H), 7.29 (m, 2H), 6.58 (d, 2H), 4.33 (d, 2H), 2.93 (s, 3H).

MS: 439.03 (M+1).

### EXAMPLE 91

### 3-(3,5-bis(trifluoromethyl)benzylamino)-2-(4-fluorophenyl)quinazolin-4(3H)-one

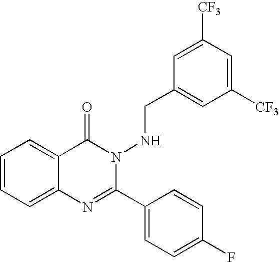

Prepared as described in Example 1.

¹H-NMR (400 MHz, CDCl₃): δ 8.34 (dd, 1H), 7.78 (m, 5H), 7.56 (sep, 1H), 7.37 (s, 2H), 7.36 (dd, 1H), 7.14 (t, 2H), 6.13 (t, 1H), 3.98 (bs, 2H).

MS: 482.21 (M+1).

### EXAMPLE 92

### 3-(4-bromobenzylamino)-2-(3,4-difluorophenyl)quinazolin-4(3H)-one

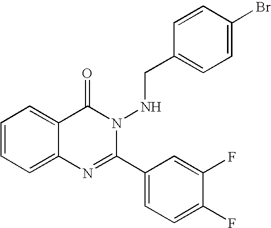

Prepared as described in Example 1.

¹H-NMR (400 MHz, CDCl₃): δ 8.26 (dd, 1H), 7.70 (m, 2H), 7.60 (m, 1H), 7.48 (m, 2H), 7.27 (dd, 2H), 7.16 (m, 1H), 6.79 (d, 1H), 5.98 (t, 1H), 3.74 (bs, 2H).

MS: 443.03 (M+1).

### EXAMPLE 93

### 3-((4-bromobenzyl)(ethyl)amino)-2-(4-fluorophenyl)quinazolin-4(3H)-one

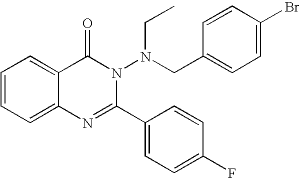

### Step 1:

In an 8 mL septa sealed vial 3-(4-bromobenzyl amino)-2-(4-fluorophenyl)quinazolin-4(3H)-one (50 mg, 0.118 mmol) was stirred in 1 mL of anhydrous THF with 200 μL of DMSO. The solution was cooled to 0° C. in an ice bath and stirred for 10 minutes. Sodium hydride (6 mg, 0.14 mmol, 60% in oil) was added carefully and stirred at 0° C. for 10 minutes. Iodoethane (11 L, 0.13 mmol) was then added to the reaction and stirred for 12 hours gradually warming to room temperature. The reaction was poured into 1 mL of water and extracted three times with ethyl acetate. The organic layers were combined, washed once with brine, dried over anhydrous sodium sulfate, filtered and concentrated. The crude material was then purified using flash chromatography eluting with 80% ethyl acetate/20% hexane to afford 43 mg (80%) of 3-((4-bromobenzyl)(ethyl)amino)-2-(4-fluorophenyl)quinazolin-4(3H)-one as an off white oil.

¹H-NMR: (CDCl₃) 8.32 (d, 1H), 7.80 (dd, 1H), 7.69 (d, 1H), 7.52 (dd, 1H), 7.30 (dd, 2H), 7.22 (d, 2H), 7.06 (dd, 2H), 6.62 (d, 2H), 4.04 (dd, 2H), 3.36 (m, 2H), 1.39 (t, 3H) ppm

MS: (452.3)

### EXAMPLE 94

### 3-((4-bromobenzyl)(propyl)amino)-2-(4-fluorophenyl)quinazolin-4(3H)-one

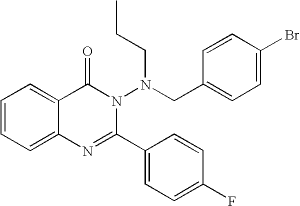

Prepared as described in Example 93.

¹H-NMR: (CDCl₃) 8.32 (d, 1H), 7.80 (dd, 1H), 7.69 (d, 1H), 7.52 (dd, 1H), 7.30 (dd, 2H), 7.22 (d, 2H), 7.06 (dd, 2H), 6.62 (d, 2H), 4.04 (dd, 2H), 3.36 (m, 2H), 1.39 (t, 3H), 0.99 (m, 2H) ppm

MS: (466.4)

### EXAMPLE 95

### 3-(4-bromobenzylamino)-2-(4-fluorophenyl)-7-hydroxy quinazolin-4(3H)-one

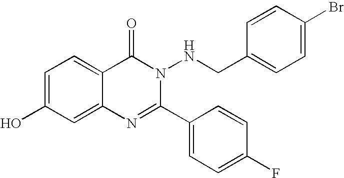

In an 8 mL septa sealed vial of 3-(4-bromobenzyl amino)-2-(4-fluorophenyl)-7-methoxyquinazolin-4(3H)-one (50 mg, 0.110 mmol) was stirred in 2 mL of collidine. Lithium iodide (74 mg, 0.550 mmol) was then added to the vial and stirred at room temperature. The vial was purged with nitrogen and heated to 160° C. for 12-36 hours. The reaction was then concentrated with a stream of nitrogen while warming at 40° C. Ethyl acetate (10 mL) was added to the crude mixture. The organics were washed three times with a concentrated solution of Cu₂SO₄ (in water). The aqueous layers were combined and washed once with ethyl acetate. The organic layers were combined, washed once with brine, dried over anhydrous sodium sulfate, filtered and concentrated. The crude material was then purified using flash chromatography eluting with 100% Ethyl Acetate to afford 34 mg (70%) of 3-(4-bromobenzylamino)-2-(4-fluorophenyl)-7-hydroxy quinazolin-4(3H)-one as an off white solid.

¹H-NMR: (DMSO) 10.60 (s, 1H), 8.01 (d, 1H), 7.62 (dd, 2H), 7.30 (d, 2H), 7.24 (dd, 2H), 7.12 (d, 1H), 7.00 (d, 1H), 6.90 (s, 1H), 6.79 (d, 1H), 6.62 (t, 1H), 3.82 (brd, 2H) ppm

MS:(440.3)

### EXAMPLE 96

### 3-(4-bromobenzylamino)-2-(3-hydroxyphenyl)quinazolin-4(3H)-one

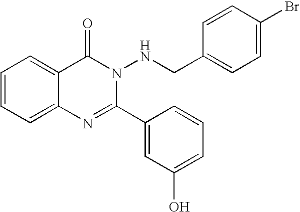

Prepared as described in Example 95.

¹H-NMR: (DMSO) 10.21 (s, 1H), 8.34 (d, 1H), 7.80 (dd, 2H), 7.58 (dd, 2H), 7.38 (m, 3H), 7.20 (s, 1H), 7.00 (dd, 1H), 6.64 (d, 2H), 6.00 (t, 1H), 3.80 (brd, 2H) ppm

MS: (422.3)

### EXAMPLE 97

### 3-(4-bromobenzylamino)-2-(4-hydroxyphenyl)quinazolin-4(3H)-one

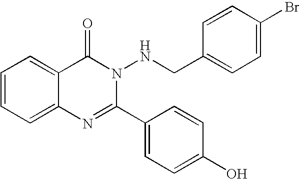

Prepared as described in Example 95.

¹H-NMR: (DMSO) 10.04 (s, 1H), 8.34 (d, 1H), 7.80 (d, 2H), 7.78 (d, 2H), 7.50 (dd, 1H), 7.36 (d, 2H), 6.69 (d, 2H), 6.68 (d, 2H), 6.04 (t, 1H), 3.80 (brd, 2H) ppm

MS: (422.3)

### EXAMPLE 98

### 3-(4-chlorobenzylamino)-2-(4-fluorophenyl)-6-hydroxyquinazolin-4(3H)-one

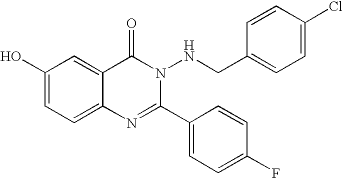

Prepared as described in Example 95.

¹H-NMR: (DMSO) 10.21 (s, 1H), 7.62 (m, 2H), 7.58 (d, 1H), 7.49 (s, 1H), 7.31 (dd, 1H), 7.22 (dd, 2H), 7.20 (dd, 2H), 6.88 (d, 2H), 6.70 (t, 1H), 3.90 (brd, 2H) ppm

MS: (395.8)

### EXAMPLE 99

### 3-(4-bromobenzylamino)-2-(4-hydroxy-3-methoxyphenyl)quinazolin-4(3H)-one

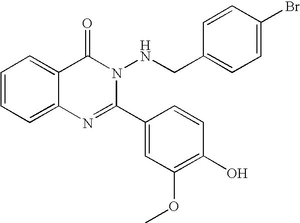

Prepared as described in Example 95.

¹H-NMR: (CDCl₃) 101(s, 1H), 8.34 (d, 1H), 7.80 (dd, 2H), 7.54 (dd, 1H), 7.39 (d, 2H), 7.34 (d, 2H), 7.22 (d, 1H), 6.84 (d, 2H), 6.08 (t, 1H), 3.84 (s, 3H), 3.80 (brd, 2H) ppm

MS: (452.3)

### EXAMPLE 100

### 3-(4-chlorobenzylamino)-2-(4-hydroxy-3-methoxyphenyl)quinazolin-4(3H)-one

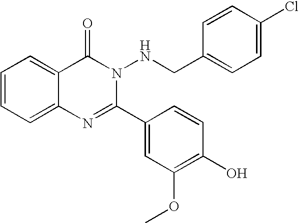

Prepared as described in Example 95.

¹H-NMR: (CDCl₃) 10.12 (s, 1H), 8.34 (d, 1H), 7.80 (dd, 2H), 7.54 (dd, 2H), 7.32 (s, 1H), 7.20 (dd, 2H), 7.02 (d, 2H), 6.98 (d, 1H), 6.12 (t, 1H), 3.84 (s, 3H), 3.80 (brd, 2H) ppm

MS: (407.9)

### EXAMPLE 101

### 4-((2-(2-fluorophenyl)-4-oxoquinazolin-3(4H)-ylamino)methyl)benzoic acid

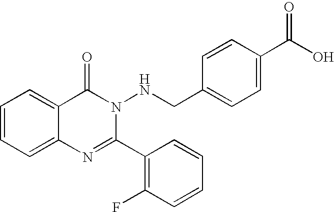

Methyl 4-((2-(2-fluorophenyl)-4-oxoquinazolin-3(4H)-ylamino)methyl)benzoate (10 mg) was stirred in 500 μL of a 1:1 solution of trifluoroacetic acid and dichloromethane in an 8 mL septa sealed vial at room temperature for three hours. The reaction was then concentrated under high vacuum to afford 8 mg (80%) of 4-((2-(2-fluorophenyl)-4-oxoquinazolin-3(4H)-ylamino)methyl)benzoic acid as a yellow solid.

¹H-NMR: (DMSO) 12.82 (s, 1H), 8.22 (d, 1H), 7.90 (dd, 2H), 7.85 (d, 1H), 7.70 (m, 2H), 7.60 (m, 1H), 7.40 (dd, 2H), 7.22 (dd, 2H), 6.99 (t, 1H), 6.82 (d, 1H), 4.02 (brd, 2H) ppm

MS: (389.3)

### EXAMPLE 102

### 4-Bromo-N-[2-(4-fluoro-phenyl)-4-oxo-4H-quinazolin-3-yl]-benzamide

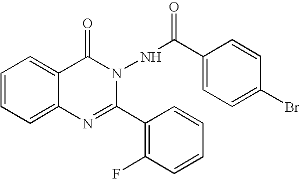

3-Amino-2-(4-fluoro-phenyl)-3H-quinazolin-4-one (100 mg, 0.39 mmol) was stirred in 1 mL of dry dichloromethane under nitrogen in an 8 mL septa sealed vial. 4-Bromobenzoyl chloride (126 mg, 0.59 mmol) was then added and stirred at room temperature for 24 hours. The reaction was diluted with 1 mL of dichloromethane, then washed twice with saturated ammonium chloride. The organics were dried over anhydrous sodium sulfate, then filtered and concentrated. Hexane was added to the residue and after 12 hours the product as a beige solid precipitated out of solution. The solids were filtered, washed with hexane and dried under high vacuum to afford 4-bromo-N-[2-(4-fluoro-phenyl)-4-oxo-4H-quinazolin-3-yl]-benzamide(67%).

¹H-NMR: (CDCl₃) 9.11 (s, 1H), 8.39 (d, 1H), 7.88 (dd, 2H), 7.84 (dd, 1H), 7.74 (dd, 1H), 7.62 (dd, 2H), 7.60 (dd, 2H), 7.44 (dd, 2H), 7.18 (dd, 1H) ppm

MS: (438.3)

### EXAMPLE 103

### 3-(benzylamino)-6-fluoro-2-(3-fluorophenyl)quinazolin-4(3H)-one

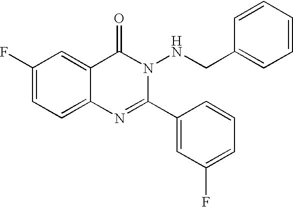

Prepared as described in Example 1.

¹H-NMR: (CDCl₃) 7.90 (d, 1H), 7.81 (dd, 1H), 7.60 (dd, 2H), 7.42 (dd, 2H), 7.35 (dd, 1H), 7.21 (m, 2H), 7.04 (m, 2H), 7.00 (dd, 1H), 6.01 (t, 1H), 3.82 (brd, 2H) ppm

MS: (363.4)

### EXAMPLE 104

### 3-(4-bromobenzylamino)-2-phenylquinazolin-4(3H)-one

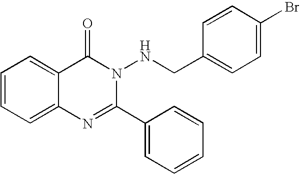

Prepared as described in Example 1.

¹H-NMR: (CDCl₃) 7.80 (d, 2H), 7.78 (dd, 1H), 7.52 (m, 3H), 7.42 (dd, 2H), 7.40 (dd, 2H), 7.26 (m, 2H), 7.21 (m, 1H), 7.00 (t, 1H), 4.62 (d, 2H) ppm

MS: (406.3)

### EXAMPLE 105

### 3-(4-bromobenzylamino)-2-(3-fluorophenyl)quinazolin-4(3H)-one

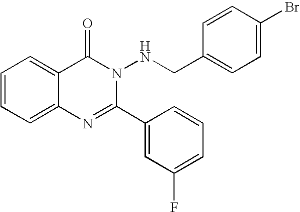

Prepared as described in Example 1.

¹H-NMR: (CDCl₃) 8.34 (d, 1H), 7.81 (dd, 2H), 7.60 (dd, 2H), 7.48 (m, 1H), 7.38 (d, 2H), 7.21 (dd, 1H), 6.82 (d, 2H), 6.01 (t, 1H), 3.82 (brd, 2H) ppm

MS: (424.5)

### EXAMPLE 106

### 3-(4-bromobenzylamino)-6-fluoro-2-(3-fluorophenyl)quinazolin-4(3H)-one

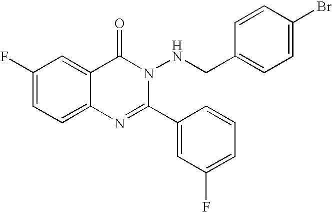

Prepared as described in Example 1.

¹H-NMR: (CDCl₃) 7.92 (d, 1H), 7.81 (dd, 1H), 7.58 (m, 2H), 7.44 (dd, 2H), 7.35 (d, 2H), 7.23 (m, 1H), 6.68 (d, 2H), 6.01 (t, 1H), 3.80 (brd, 2H) ppm

MS: (442.5)

### EXAMPLE 107

### 3-(4-fluoro-3-(trifluoro methyl)benzylamino)-2-(3-fluorophenyl)quinazolin-4(3H)— one

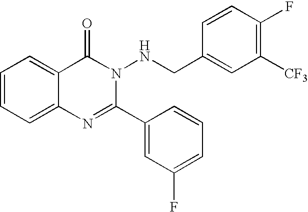

Prepared as described in Example 1.

¹H-NMR: (CDCl₃) 8.40 (d, 1H), 7.82 (dd, 1H), 7.79 (dd, 1H), 7.60 (m, 2H), 7.42 (dd, 2H), 7.26 (m, 2H), 7.14 (m, 1H), 7.10 (dd, 1H), 7.01 (t, 1H), 5.22 (d, 2H) ppm

MS: (431.4)

### EXAMPLE 108

### 3-(4-chlorobenzylamino)-7-fluoro-2-(4-fluorophenyl)quinazolin-4(3H)-one

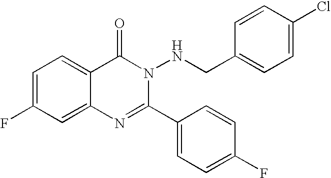

Prepared as described in Example 1.

¹H-NMR: (CDCl₃) 8.34 (d, 1H), 7.81 (d, 2H), 7.40 (d, 1H), 7.28 (dd, 2H), 7.18 (dd, 2H), 7.12 (m, 1H), 6.84 (d, 2H), 6.01 (t, 1H), 3.79 (brd, 2H) ppm

MS: (397.8)

### EXAMPLE 109

### 3-(4-bromobenzylamino)-2-(3-chloro-4-methylphenyl)quinazolin-4(3H)-one

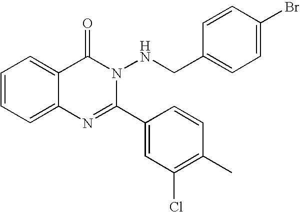

Prepared as described in Example 1.

¹H-NMR: (CDCl₃) 8.31 (d, 1H), 7.81 (d, 2H), 7.79 (dd, 1H), 7.62 (d, 1H), 7.52 (dd, 1H), 7.32 (d, 2H), 7.30 (m, 1H), 6.62 (d, 2H), 6.02 (t, 1H), 3.80 (brd, 2H) ppm

MS: (454.8).

### EXAMPLE 110

### 2-(3-chloro-4-methylphenyl)-3-(4-chlorobenzylamino)quinazolin-4(3H)-one

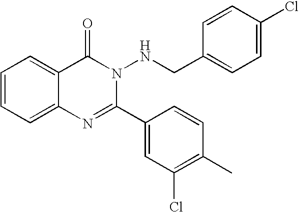

Prepared as described in Example 1.

¹H-NMR: (CDCl₃) 8.31 (d, 1H), 7.82 (d, 2H), 7.78 (dd, 1H), 7.62 (d, 1H), 7.54 (dd, 1H), 7.30 (dd, 1H), 7.19 (d, 2H), 6.92 (d, 2H), 6.02 (t, 1H), 3.82 (brd, 2H) ppm

MS: (410.3)

### EXAMPLE 111

### 3-(4-bromobenzylamino)-2-(4-fluorophenyl)-5-methylquinazolin-4(3H)-one

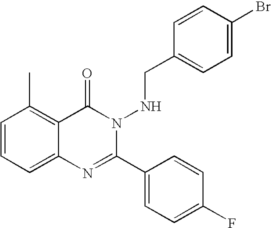

Prepared as described in Example 1.

¹H-NMR (400 MHz, CDCl₃): δ 7.81 (q, 2H), 7.61 (m, 2H), 7.32 (d, 2H), 7.28 (d, 1H), 7.14 (t, 2H), 6.85 (d, 2H), 6.04 (t, 1H), 3.74 (bs, 2H), 2.93 (s, 3H).

LCMS: 439.46 (M+1)

### EXAMPLE 112

### 3-(4-bromobenzylamino)-2-(4-fluorophenyl)-5-methylquinazolin-4(3H)-one

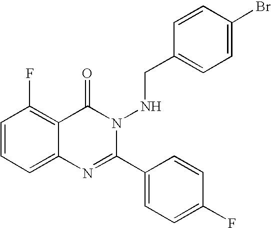

Prepared as described in Example 1.

¹H-NMR (400 MHz, CDCl₃): δ 7.82 (q, 2H), 7.72 (sext, 1H), 7.55 (d, 1H), 7.32 (d, 2H), 7.15 (m, 3H), 6.85 (d, 2H), 6.03 (t, 1H), 3.76 (bs, 2H).

LCMS: 443.45 (M+1).

### EXAMPLE 113

### 3-(4-bromobenzylamino)-2-(4-fluorophenyl)-8-hydroxyquinazolin-4(3H)-one

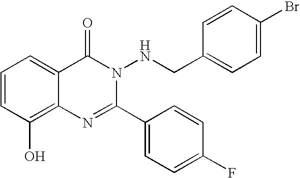

Prepared as described in Example 95.

### EXAMPLE 114

### 3-(4-chlorobenzylamino)-2-(4-fluorophenyl)-8-hydroxyquinazolin-4(3H)-one

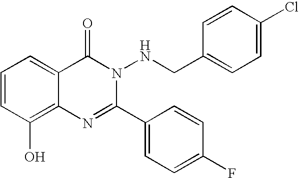

Prepared as described in Example 95.

### EXAMPLE 115

### 3-(4-bromobenzylamino)-5-fluoro-2-(4-fluorophenyl)quinazolin-4(3H)— one

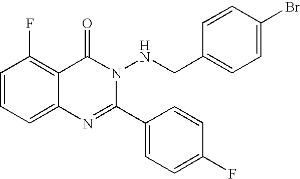

Prepared as described in Example 1.

### EXAMPLE 116

### 3-(4-bromobenzylamino)-2-(6-methoxypyridin-3-yl)quinazolin-4(3H)— one

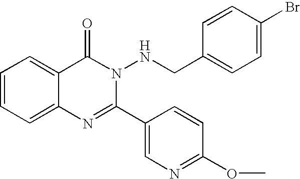

Prepared as described in Example 1.

### EXAMPLE 117

### 3-(4-bromobenzylamino)-2-(6-hydroxypyridin-3-yl)quinazolin-4(3H)-one

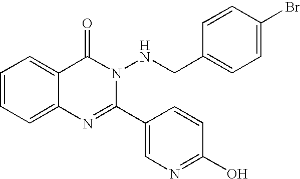

Prepared as described in Example 95.

### EXAMPLE 118

### 3-(4-bromobenzylamino)-2-(3-hydroxy-4-methoxyphenyl)quinazolin-4(3H)-one

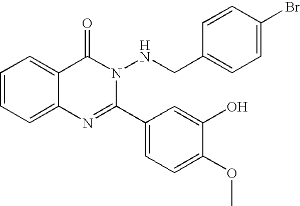

Prepared as described in Example 95.

### EXAMPLE 119

### 3-(4-chlorobenzylamino)-2-(3-hydroxy-4-methoxyphenyl)quinazolin-4(3H)— one

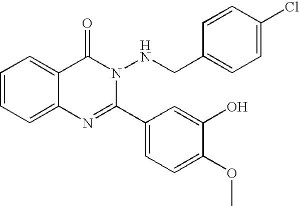

Prepared as described in Example 95.

The activity of the compounds in Examples 1-119 as TGR5 modulators is illustrated in the following assays.

### Biological Activity Assay

cAMP Production Assay:

HEK293 cells stably expressing TGR5 (HEK293-TGR5) were established by stably transfecting HEK-293 cells with an expression vector (pcDNA 3.1, Invetrogen) inserted with human TGR5 cDNA using Fugene6 (Roche, Indianapolis, Ind.) according to conventional methods. Cells were grown in DMEM (invitrogen, Carlsbad, Calif.) supplemented with 10% FBS, 1% penicillin/streptomycin under geneticin selection. The presence of TGR5 transcripts in these cells was confirmed using branched DNA (bDNA, Genospectra, Inc., Fremont Calif.) following the manufacturer's protocol and using specific probes for human TGR5. cAMP production assay was performed in high throughput 1536 well format using LANCE cAMP detection kit (Perkin Elmer Inc., Boston, Mass.) according to the manufacturer's protocol. Briefly, HEK293-TGR5 cells were harvested using non-enzymatic cell dissociation buffer (Invitrogen, Carlsbad, Calif.) and suspended in DMEM supplemented with 0.1% FBS at a density of 800,000 cells/ml. Alexa antibody was added to the cell suspension, and 4 ul of the mixture was dispensed in white opaque tissue culture treated Greiner 1536 well plates (USA Scientific, Inc., Ocala, Fla.). After an overnight incubation at 37 C in an atmosphere of 10% CO2 and 95% humidity, 1 ul of 5 mM IBMX (Sigma, St. Louis, Mo.) solution in DMEM was dispensed for a final concentration of 1 mM. Cells were then stimulated with test compounds for 30 minutes, after which time 5 ul of detection reagent was added and incubated for 1-7 hrs at room temperature. TR-FRET signal was detected using the Viewlux (Perkin Elmer Inc., Boston Mass.). EC₅₀ values were determined using Graph Pad Prizm analysis (GraphPad Software, Inc). The EC₅₀ values for a wide range of bile acids generated from this assay were in agreement with the values published in the scientific literature. None of the compounds induced cAMP in HEK-293 cells that were transfected with an empty vector alone, confirming a TGR5 mechanism of action for cAMP production. The symbol (+) denotes an EC₅₀ value of ≦10 μM while the symbol (−) denotes an EC₅₀ value of >10 μM (see Table 1).

### Glucagon-like Peptide-1 (GLP-1) Secretion Assay:

NCI-H716 cells, human enteroendocrine (ATCC# CCL-251) have been shown to express TGR5 (Maruyama T. et al, BBRC 298, 714-719, 2002) and to secrete GLP-1 in response to nutrients such as fatty acids and meat hydrolysate (Reimer R. et al, Endocrinology 142: 4522-4528). Cells were cultured and maintained in RPMI1640 (Invitrogen, Carlsbad, Calif.) supplemented with 10% FBS, 1% penicillin/streptomycin and 1% sodium pyruvate. Two days before GLP-1 secretion assays, 1×10⁵ cells were seeded in 96 well culture plates coated with Matrigel (BD Biosciences, Bedford, Mass.). On the day of experiment, the culture medium was removed and the cells were washed twice with KRB buffer (116 mM NaCl, 4.7 mM KCl, 2.5 mM CaCl2, 25 mM NaHCO3, 1.2 mM K2HPO4, 1.2 mM MgCl2, 25 mM HEPES, 0.2% BSA, pH 7.3). Test compounds (or DMSO control, 0.1% final concentration) were added to the cells in the same buffer and were incubated at 37 C for 2 hrs. Supernatants were then collected, centrifuged at 2000 rpm for 5 min to remove cell debris and were used to measure GLP-1 by ELISA (Linco Research, Inc., St. Charles, Mo.). This kit measures the biologically active GLP-1 (7-36 amide and 7-37). All compounds were tested at 10 μM. The activity of the compounds is expressed as (+ or −) based of the ability of the compound to induce a statistically significant GLP-1 secretion above the DMSO vehicle control at 10 μM. The authors theorize, although they do not wish to be held to this theory, that the compounds which score positive in this assay are more likely to induce GLP-1 secretion in vivo, and therefore possess a better therapeutic profile for the claimed indications (see Table 1).

TABLE 1
In Vitro Biological Activity Assays

| | | GLP-1 Secretion: |
|---|---|---|
| | cAMP | (+): statistically significant |
| | Production in | above the DMSO control at |
| | 293-TGR5 | 10 μM |
| | Cells | (−): not statistically |
| | EC₅₀: | significant above the |
| Example | (+): ≦10 μM; | DMSO control at 10 μM; |
| No. | (−): >10 μM | NT = not tested |
| 1 | + | − |
| 2 | − | NT |
| 3 | + | NT |
| 4 | + | NT |
| 5 | + | NT |
| 6 | + | NT |
| 7 | + | NT |
| 8 | + | NT |
| 9 | + | NT |
| 10 | + | NT |
| 11 | − | NT |
| 12 | + | NT |
| 13 | + | NT |
| 14 | + | NT |
| 15 | + | NT |
| 16 | + | NT |
| 17 | + | NT |
| 18 | + | NT |
| 19 | − | NT |
| 20 | + | NT |
| 21 | + | NT |
| 22 | + | NT |
| 23 | + | NT |
| 24 | + | NT |
| 25 | + | NT |
| 26 | − | NT |
| 27 | + | NT |
| 28 | − | NT |
| 29 | − | NT |
| 30 | − | NT |
| 31 | + | NT |
| 32 | + | NT |
| 33 | − | NT |
| 34 | − | NT |
| 35 | − | NT |
| 36 | − | NT |
| 37 | − | NT |
| 38 | + | NT |
| 39 | − | NT |
| 40 | + | NT |
| 41 | + | NT |
| 42 | − | NT |
| 43 | + | NT |
| 44 | + | NT |
| 45 | + | NT |
| 46 | + | NT |
| 47 | + | NT |
| 48 | + | NT |
| 49 | + | NT |
| 50 | + | NT |
| 51 | + | NT |
| 52 | + | NT |
| 53 | + | NT |
| 54 | + | NT |
| 55 | + | NT |
| 56 | + | NT |
| 57 | + | NT |
| 58 | + | NT |
| 59 | + | NT |
| 60 | + | NT |
| 61 | + | NT |
| 62 | − | NT |
| 63 | + | NT |
| 64 | + | NT |
| 65 | + | NT |
| 66 | + | NT |
| 67 | + | NT |
| 68 | + | NT |
| 69 | + | NT |
| 70 | + | NT |
| 71 | + | NT |
| 72 | + | NT |
| 73 | + | NT |
| 74 | + | NT |
| 75 | + | NT |
| 76 | + | NT |
| 77 | + | NT |
| 78 | + | NT |
| 79 | + | NT |
| 80 | + | NT |
| 81 | + | NT |
| 82 | − | NT |
| 83 | − | NT |
| 84 | − | NT |
| 85 | + | NT |
| 86 | + | NT |
| 87 | + | NT |
| 88 | + | NT |
| 89 | + | NT |
| 90 | + | NT |
| 91 | + | NT |
| 92 | + | NT |
| 93 | + | NT |
| 94 | + | NT |
| 95 | + | NT |
| 96 | + | NT |
| 97 | + | NT |
| 98 | + | NT |
| 99 | + | NT |
| 100 | + | NT |
| 101 | − | NT |
| 102 | − | NT |
| 103 | + | NT |
| 104 | + | NT |
| 105 | + | NT |
| 106 | + | NT |
| 107 | + | NT |
| 108 | + | NT |
| 109 | + | NT |
| 110 | + | NT |
| 111 | + | NT |
| 112 | + | NT |
| 113 | + | NT |
| 114 | + | NT |
| 115 | + | NT |
| 116 | + | NT |
| 117 | + | NT |
| 118 | + | NT |
| 119 | + | NT |

### In Vivo Assays

### Dosages

Vehicle is CMC-Tween: 0.5% CMC+0.25% Tween 20 at an appropriate ml/kg. Dextrose dose per mouse=2 g dextrose/kg made from 0.5 g/ml stock dextrose solution. Compound 1 dose per mouse=30 mg/kg in CMC/Tween vehicle made from 4 mg/mL stock solution by adding 50 μL DMSO to 20 mg Compound 1, stirring to make a paste, adding 1 ml CMC/Tween with stirring, sonicating for 8 minutes on energy 05, and adjusting to 5 ml with CMC/Tween.

### Glucose Tolerance Test (GTT)/Insulin Test

Mice (5 per group) were fasted at 6 am for an experiment at noon. Blood glucose was measured from tail nick blood using an Accucheck—Compact Monitoring Kit (Roche Diagnostics, Indianapolis Ind.). Baseline (time=−15 min) blood glucose concentration was determined for all mice and tail nick blood samples were collected. 2 g/kg dextrose (Hospira, Lake Forest, Ill.) was administered orally at time 0. Blood samples were collected from the following mice via tail nick blood draw (˜100 μL) into EDTA microtainer tubes at the following time points: −15, 0, 3, 6, 9, 12, 15, 20, 30 and 120 min. Terminal blood samples were collected from portal vein draws in three mice at t=20. Blood plasma was harvested from blood collected in EDTA microvettes by centrifugation (10 min at 8.6 rpm). Plasma was stored in −80 C freezer to be analyzed for other chemistry measurements.

### Insulin ELISA Assay

A Mercodia ultrasensitive mouse insulin ELISA kit (Mercodia AB, Uppsala, Sweden) was used in a 96-well coated plate. All reagents and samples were brought to room temperature before use, Calibrator concentrations were 0.07, 0.13, 0.25, 0.63, 1.9, 5.2, 13.0, and 64.0 μg/L, and a calibration curve was prepared for each assay run. To 25 μL calibrator 0 in each well was added 5 μL calibrators and 5 μL of samples/standards in duplicate, then 50 μL dilute enzyme conjugate 11× in enzyme conjugate buffer. This was incubated on a shaker for 2 hours at room temperature (18-25° C.), and each well was washed 5 times in 350 μL wash buffer and aspirated completely. After final wash, plate was inverted and tapped firmly against absorbent paper. 200 μL substrate TMB was added to each well and incubated for 30 minutes. 50 μL stop solution was added to each well, and the plate was placed on the shaker for about 5 seconds to ensure mixing of substrate and stop Solution. Absorbance was measured at 450 nm.

### GLP-1 Protocol

Mice (3 per group) were fasted at 6 am for an experiment at noon. Blood glucose was measured from tail nick blood using an Accucheck—Compact Monitoring Kit (Roche Diagnostics, Indianapolis Ind.). Baseline (time=−15 min) blood glucose concentration was determined for all mice. 2 g/kg dextrose (Hospira, Lake Forest, Ill.) was administered orally at time 0. Terminal blood samples were collected from the mice via portal vein draw (˜500 μL) into EDTA microtainer tubes at the following time points: −15, 0, 3, 6, 9, 12, 15, 20, 30 and 120 min. The microtainer tube contained both 5 μL of a DPP4 inhibitor (Linco research, St. Charles, Mo.) and 5 μL of 10× conc. of a Protease inhibitor cocktail (Complete mini inhibitor cocktail, Roche Diagnostics, Mannheim, Germany). Blood plasma was harvested from blood collected in EDTA microvettes by centrifugation (10 min at 8.6 rpm). Plasma was stored in −80 C freezer to be analyzed for other chemistry measurements.

### GLP-1 Assay

A GLP-1(Human, Mouse, Rat) ELA kit was used (ALPCO Diagnostics, Salem, N.H., Cat # 48-GLP-1-160) was used in a 96-well plate. After 3×350 μL wash of each well, 40 μL of labeled antigen (with 6 mL of distilled water per vial) was added to each well, then 30 μL of samples/standards (these latter made from 50 ng/ml standard solution serially diluted to get 16.7, 5.6, 1.9, 0.6, 0.21, 0.07 ng/mL standards; buffer solution is used as 0 ng/mL), then 40 μL of GLP-1 antibody. Plate was covered with adhesive sealer and incubated at 4° C. overnight. Sealer was removed and wells washed 4× with 350 μL washing solution. 100 μL/well of diluted SA-HRP solution (120 μL of SA-HRP 12 μL diluent for SA-HRP, well mixed just before use) was added to each well, and the plate covered with adhesive sealer and incubate at room temperature for 1 hr on a plate shaker. Sealer was removed and wells washed 4× with 350 μL washing solution. 100 μL/well of substrate solution (1 OPD tablet in 12 mL of substrate buffer, made just before use) was added to each well and the plate again covered with adhesive sealer and incubated at room temperature for 30 min on a plate shaker. 100 μl/well stop solution was added to each well to stop reaction, optical absorbance of wells read at 490 nm, and results calculated.

TABLE 2
Glucose Tolerance Test
Glucose Disposal

| | Plasma Glucose, mg/dL: | | Plasma Glucose, mg/dL: | |
|---|---|---|---|---|
| | Example 1, 30 mg/kg | | Vehicle | |
| Time, min | Mean | SE | Mean | SE |
| −15 | 170.0 | 4.39 | 124.0 | 17.62 |
| 0 | 220.4 | 25.94 | 195.0 | 23.99 |
| 3 | 272.2 | 34.06 | 207.2 | 23.15 |
| 6 | 332.6 | 35.17 | 232.8 | 16.13 |
| 9 | 398.2 | 33.28 | 268.8 | 19.58 |
| 12 | 427.2 | 43.26 | 332.2 | 16.40 |
| 15 | 430.0 | 41.03 | 366.2 | 28.09 |
| 20 | 425.9 | 36.74 | 473.8 | 32.60 |
| 30 | 354.6 | 33.29 | 519.0 | 53.00 |
| 60 | 294.2 | 26.48 | 488.4 | 38.36 |
| 120 | 213.2 | 23.29 | 261.2 | 15.20 |

TABLE 3
Glucose Tolerance Test, Area Under the Curve
Glucose Disposal

| | |
|---|---|
| Plasma Glucose, AUC: | Plasma Glucose, AUC: |
| Example 1, 30 mg/kg | Vehicle |

| Time, min | Mean | SE | Mean | SE |
|---|---|---|---|---|
| 0-120 | 16400 | 5117 | 34280 | 1158 |

TABLE 4
Glucose-Stimulated Insulin Secretion
Glucose-Stimulated Insulin Secretion

| | Plasma Insulin, ng/mL: | | Plasma Insulin, ng/mL: | |
|---|---|---|---|---|
| | Example 1, 30 mg/kg | | Vehicle | |
| Time, min | Mean | SE | Mean | SE |
| −15 | 1.78 | 0.51 | 1.20 | 0.24 |
| 0 | 1.56 | 0.26 | 0.75 | 0.39 |
| 3 | 2.35 | 0.58 | 0.66 | 0.24 |
| 6 | 3.24 | 0.64 | 0.85 | 0.32 |
| 9 | 3.94 | 0.74 | 0.75 | 0.20 |
| 12 | 4.18 | 0.74 | 1.70 | 0.33 |
| 15 | 4.05 | 0.59 | 1.36 | 0.49 |
| 20 | 3.79 | 0.47 | 2.56 | 0.39 |
| 30 | 1.82 | 0.20 | 1.93 | 0.44 |
| 60 | 1.23 | 0.32 | 1.29 | 0.33 |
| 120 | 1.17 | 0.30 | 1.34 | 0.42 |

TABLE 5
Glucose-Stimulated Insulin Secretion, Area Under the Curve
Glucose-Stimulated Insulin Secretion

| | |
|---|---|
| Plasma Insulin, AUC: | Plasma Insulin, AUC: |
| Example 1, 30 mg/kg | Vehicle |

| Time, min | Mean | SE | Mean | SE |
|---|---|---|---|---|
| 0-15 | 35.15 | 9.834 | 6.204 | 1.42 |
| 0-30 | 68.39 | 14.3 | 29.61 | 5.211 |

TABLE 4
GLP-1 Secretion
GLP-1 Secretion

| | |
|---|---|
| Plasma GLP-1, ng/mL: | Plasma GLP-1, ng/mL: |
| Example 1, 30 mg/kg | Vehicle |

| Time, min | Mean | SE | Mean | SE |
|---|---|---|---|---|
| −15 | 6.30 | 0.24 | 7.18 | 0.30 |
| 20 | 9.61 | 0.77 | 5.73 | 0.15 |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treatment of a TGR5-mediated disease comprising the administration, to a patient in need thereof, of a therapeutically effective amount of a compound having structural Formula II: 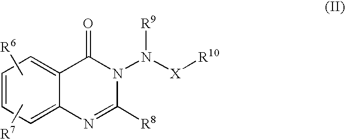
or a salt, ester, or prodrug thereof, wherein:
X is C₁₋₂alkyl, optionally substituted with one or more substituents selected from the group consisting of C₁₋₃alkyl, halogen, and oxo;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl, and amino; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

2. The method as recited in claim 1 wherein:
R⁶ and R⁷ are independently selected from the group consisting of hydrogen, halogen, alkoxy, alkyl, haloalkyl, perhaloalkyl, and perhaloalkoxy;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino;
R⁹ is selected from the group consisting of hydrogen and lower alkyl; and
R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

3. The method as recited in claim 2 wherein:
X is CH₂;
R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino;
R⁹ is hydrogen; and
R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

4. The method as recited in claim 3, wherein:
R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

5. The method as recited in claim 4, wherein:
R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

6. The method as recited in claim 5, wherein R¹⁰ is phenyl, which is substituted in the para position by halogen.

7. The method as recited in claim 1, wherein said compound having structural Formula II is selected from the group consisting of Examples 1 to 119.

8. The method as recited in claim 1 wherein said disease is a metabolic disease.

9. The method as recited in claim 8 wherein said disease is selected from the group consisting of inadequate glucose tolerance, insulin resistance, type I diabetes, and type II diabetes.

10. The method as recited in claim 8 further comprising the administration of another therapeutic agent.

11. The method as recited in claim 10, wherein said agent is selected from the group consisting of insulin, metformin, Glipizide, glyburide, Amaryl, gliclazide, meglitinides, nateglinide, repaglinide, pramlintide, PTP-112, SB-517955, SB-4195052, SB-216763, N,N-57-05441, N,N-57-05445, GW-0791, AGN-¹⁹4²⁰4, T-1095, BAY R3401, acarbose, miglitol, voglibose, Exendin-4, DPP728, LAF237, vildagliptin, BMS477118, PT-100, GSK-823093, PSN-9301, T-6666, SYR-322, SYR-619, Liraglutide, CJC-1134-PC, naliglutide, MK-0431, saxagliptin, GSK23A, pioglitazone, rosiglitazone, AVE2268, GW869682, GSK189075, APD668, PSN-119-1, PSN-821, rosuvastatin, atrovastatin, simvastatin, lovastatin, pravastatin, fluvastatin, cerivastatin, rosuvastatin, pitavastatin, fenofibrate, benzafibrate, clofibrate, gemfibrozil, Ezetimibe, eflucimibe, CP-529414, CETi-1, JTT-705, cholestyramine, colestipol, niacin, implitapide, (R)-1-{4-[5-methyl-2-(4-trifluoromethyl-phenyl)-oxazol-4-ylmethoxy]-benzenesulfonyl}2,3-dihydro-1H-indole-2-carboxylic acid, and GI-262570.

12. The method as recited in claim 1 wherein said disease is associated with perturbed bile acid metabolism.

13. The method as recited in claim 12 further comprising the administration of another therapeutic agent.

14. The method as recited in claim 1 wherein said disease is an inflammatory disease.

15. The method as recited in claim 14 wherein said disease is selected from the group consisting of rheumatoid arthritis, ulcerative colitis, and inflammatory bowel disease.

16. The method as recited in claim 14 further comprising the administration of another therapeutic agent.

17. The method as recited in claim 16, wherein said agent is selected from the group consisting of betamethasone dipropionate, betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream, guaifenesin, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin, clonidine, codeine, loperamide, tramadol, morphine, fentanyl, oxycodone, hydrocodone, levorphanol, butorphanol, menthol, oil of wintergreen, camphor, eucalyptus oil, turpentine oil, acetaminophen, infliximab, etanerecept, infliximab, and capsaicin.

18. The method as recited in claim 1 wherein said disease is obesity.

19. The method as recited in claim 18 wherein said method achieves an effect selected from the group consisting of decreasing body weight and controlling weight gain.

20. The method as recited in claim 18 further comprising the administration of another therapeutic agent.

21. The method as recited in claim 20, wherein said agent is selected from the group consisting of sibutramine, bromocriptine, Orlistat, rimonabant, Axokine, and bupropion.

22. A pharmaceutical composition comprising a pharmaceutically acceptable carrier together with a compound having structural Formula II: 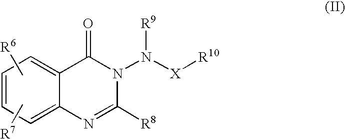
or a salt, ester, or prodrug thereof, wherein:
X is CH₂;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and
R¹⁰ is selected from the group consisting of aryl, heteroaryl, cycloalkyl, and heterocycloalkyl, substituted with at least one substituent selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

23. The pharmaceutical composition as recited in claim 22, wherein:
R⁶ and R⁷ are independently selected from the group consisting of hydrogen, halogen, alkoxy, alkyl, haloalkyl, perhaloalkyl, and perhaloalkoxy;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino;
R⁹ is selected from the group consisting of hydrogen and lower alkyl; and
R¹⁰ is selected from the group consisting of aryl, heteroaryl, cycloalkyl, and heterocycloalkyl, substituted with at least one substituent selected from the group consisting of halogen, alkyl, haloalkyl, perhaloalkyl, alkoxy, perhaloalkoxy, carboxyl, and cyano.

24. The pharmaceutical composition as recited in claim 23 wherein:
R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino;
R⁹ is hydrogen; and
R¹⁰ is selected from the group consisting of lower aryl, lower heteroaryl, lower cycloalkyl, and lower heterocycloalkyl, substituted with at least one substituent selected from the group consisting of halogen, alkyl, haloalkyl, perhaloalkyl, alkoxy, perhaloalkoxy, carboxyl, and cyano.

25. The pharmaceutical composition as recited in claim 24 wherein:
R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of phenyl, 5-membered monocyclic heteroaryl, and 6-membered monocyclic heteroaryl, substituted with at least one substituent selected from the group consisting of halogen, alkyl, haloalkyl, perhaloalkyl, alkoxy, perhaloalkoxy, carboxyl, and cyano.

26. The pharmaceutical composition as recited in claim 25 wherein:
R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is phenyl, substituted with at least one substituent selected from the group consisting of halogen, alkyl, haloalkyl, perhaloalkyl, alkoxy, perhaloalkoxy, carboxyl, and cyano.

27. The pharmaceutical composition as recited in claim 26 wherein R¹⁰ is phenyl, which is substituted in the para position by halogen.

28. A pharmaceutical composition comprising a pharmaceutically acceptable carrier together with a compound having structural Formula II: 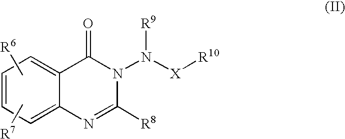
or a salt, ester, or prodrug thereof, wherein:
X is C₁₋₂alkyl, optionally substituted with one or more substituents selected from the group consisting of C₁₋₃alkyl, halogen, and oxo;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of bicyclic aryl, 5-membered nitrogen-containing heteroaryl, 6-membered heteroaryl, bicyclic heteroaryl, saturated or partially unsaturated heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl, and amino; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

29. The pharmaceutical composition as recited in claim 28, wherein:
X is CH₂;
R⁶ and R⁷ are independently selected from the group consisting of hydrogen, halogen, alkoxy, alkyl, haloalkyl, perhaloalkyl, and perhaloalkoxy;
R⁸ is selected from the group consisting of bicyclic aryl, 5-membered nitrogen-containing heteroaryl, 6-membered heteroaryl, bicyclic heteroaryl, saturated or partially unsaturated heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino;
R⁹ is selected from the group consisting of hydrogen and lower alkyl; and
R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

30. The pharmaceutical composition as recited in claim 29 wherein:
R⁸ is selected from the group consisting of bicyclic aryl, 5-membered nitrogen-containing heteroaryl, 6-membered heteroaryl, bicyclic heteroaryl, lower saturated or partially unsaturated heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino;
R⁹ is hydrogen; and
R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

31. The pharmaceutical composition as recited in claim 30 wherein:
R⁸ is selected from the group consisting of 5-membered nitrogen-containing monocyclic heteroaryl and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

32. The pharmaceutical composition as recited in claim 31 wherein R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

33. The pharmaceutical composition as recited in claim 32 wherein R¹⁰ is phenyl, which is substituted in the para position by halogen.

34. A pharmaceutical composition comprising a pharmaceutically acceptable carrier together with a compound having structural Formula II: 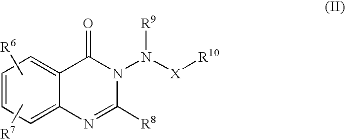
or a salt, ester, or prodrug thereof, wherein:
X is CH₂;
R⁶ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁷ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is hydrogen; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two substituents may be joined together to form a fused five-, six-, or seven-membered carbocyclic or heterocyclic ring comprising zero to three heteroatoms.

35. The pharmaceutical composition as recited in claim 34, wherein:
R⁶ is selected from the group consisting of halogen, alkoxy, alkyl, haloalkyl, perhaloalkyl, and perhaloalkoxy;
R⁷ is selected from the group consisting of hydrogen, halogen, alkoxy, alkyl, haloalkyl, perhaloalkyl, and perhaloalkoxy;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

36. The pharmaceutical composition as recited in claim 35 wherein:
R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

37. The pharmaceutical composition as recited in claim 36 wherein:
R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

38. The pharmaceutical composition as recited in claim 37 wherein:
R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

39. The pharmaceutical composition as recited in claim 38 wherein R¹⁰ is phenyl, which is substituted in the para position by halogen.

40. A pharmaceutical composition comprising a pharmaceutically acceptable carrier together with a compound selected from the group consisting of Examples 1 to 119.

41. A compound having structural Formula II: 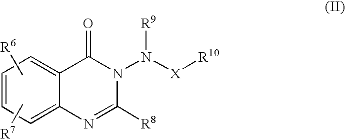
or a salt, ester, or prodrug thereof, wherein:
X is CH₂;
R⁶ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁷ is selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is hydrogen; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

42. The compound as recited in claim 41, wherein:
R⁶ is selected from the group consisting of halogen, alkoxy, alkyl, haloalkyl, perhaloalkyl, and perhaloalkoxy;
R⁷ is selected from the group consisting of hydrogen, halogen, alkoxy, alkyl, haloalkyl, perhaloalkyl, and perhaloalkoxy;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of aryl, heteroaryl, cycloalkyl, and heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

43. The compound as recited in claim 42, wherein:
R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of lower aryl, lower heteroaryl, lower cycloalkyl, and lower heterocycloalkyl, any of which may be optionally substituted with one or more substituents selected from the group consisting of halogen, alkyl, haloalkyl, perhaloalkyl, alkoxy, perhaloalkoxy, carboxyl, and cyano.

44. The compound as recited in claim 43, wherein:
R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, carboxyl, and cyano.

45. The compound as recited in claim 44, wherein:
R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino; and
R¹⁰ is phenyl, which may be optionally substituted with one or more substituents selected from the group consisting of halogen, alkyl, haloalkyl, perhaloalkyl, alkoxy, perhaloalkoxy, carboxyl, and cyano.

46. The compound as recited in claim 45, wherein R¹⁰ is phenyl, which is substituted in the para position by halogen.

47. A compound having structural Formula II: 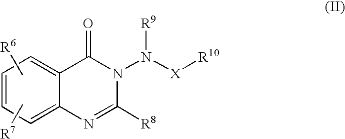
or a salt, ester, or prodrug thereof, wherein:
X is CH₂;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and
R¹⁰ is para-chlorophenyl.

48. The compound as recited in claim 47, wherein:
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R⁹ is selected from the group consisting of hydrogen and lower alkyl.

49. The compound as recited in claim 48, wherein:
R⁸ is selected from the group consisting of lower aryl, lower heteroaryl, lower cycloalkyl, lower heterocycloalkyl, lower arylalkyl, lower heteroarylalkyl, lower cycloalkylalkyl, and lower heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, and dialkylamino; and
R⁹ is hydrogen.

50. The compound as recited in claim 49, wherein:
R⁸ is selected from the group consisting of phenyl, 5-membered monocyclic heteroaryl, and 6-membered monocyclic heteroaryl, any of which may be optionally substituted with one or more substituents selected from the group consisting of halogen, alkyl, haloalkyl, perhaloalkyl, alkoxy, perhaloalkoxy, cyano, hydroxyl, and dialkylamino.

51. The compound as recited in claim 50, wherein:
R⁸ is phenyl, which may be optionally substituted with one or more substituents selected from the group consisting of halogen, alkyl, haloalkyl, perhaloalkyl, alkoxy, perhaloalkoxy, cyano, hydroxyl, and dialkylamino.

52. A compound selected from the group consisting of Examples 7 to 119.

53. A compound for use as a medicament, wherein said compound has structural Formula II: 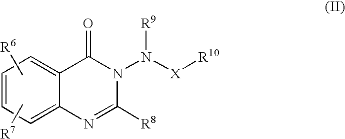
or a salt, ester, or prodrug thereof, wherein:
X is C₁₋₂alkyl, optionally substituted with one or more substituents selected from the group consisting of C₁₋₃alkyl, halogen, and oxo;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising of zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

54. A compound for use in the manufacture of a medicament for the prevention or treatment of a disease or condition ameliorated by the modulation of TGR5, wherein said compound compound has structural Formula II: 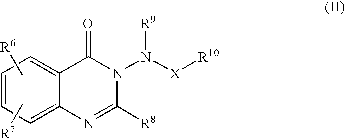
or a salt, ester, or prodrug thereof, wherein:
X is C₁₋₂alkyl, optionally substituted with one or more substituents selected from the group consisting of C₁₋₃alkyl, halogen, and oxo;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

55. A method of modulation of TGR5 comprising contacting TGR5 with a compound having structural Formula II: 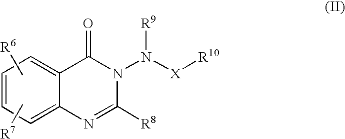
or a salt, ester, or prodrug thereof, wherein:
X is C₁₋₂alkyl, optionally substituted with one or more substituents selected from the group consisting of C₁₋₃alkyl, halogen, and oxo;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms.

56. A method for achieving an effect in a patient comprising the administration of a therapeutically effective amount of a compound having structural Formula II: 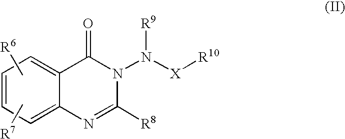
or a salt, ester, or prodrug thereof, wherein:
X is C₁₋₂alkyl, optionally substituted with one or more substituents selected from the group consisting of C₁₋₃alkyl, halogen, and oxo;
R⁶ and R⁷ are independently selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or R⁶ and R⁷ may be joined together to form a fused five-, six-, or seven-membered carbocyclic or heterocyclic ring comprising zero to three heteroatoms;
R⁸ is selected from the group consisting of aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
R⁹ is selected from the group consisting of hydrogen, alkyl, acyl, aryl, heteroaryl, cycloalkyl, heterocycloalkyl, arylalkyl, heteroarylalkyl, cycloalkylalkyl, and heterocycloalkylalkyl, any of which may be optionally substituted with one or more substituents selected from the group consisting of halogen, alkyl, haloalkyl, perhaloalkyl, alkoxy, perhaloalkoxy, cyano, hydroxyl, carboxyl, acyl-, and amino; and
R¹⁰ is selected from the group consisting of aryl, heteroaryl, cycloalkyl, and heterocycloalkyl, any of which may be optionally substituted with one or more substituents selected from the group consisting of alkyl, alkenyl, alkynyl, acyl, heteroalkyl, heterocycloalkyl, haloalkyl, perhaloalkyl, perhaloalkoxy, cycloalkyl, aryl, aryloxy, alkoxy, haloalkoxy, acyloxy, carbonyl, carboxyl, alkylcarbonyl, cyano, hydrogen, halogen, hydroxy, amino, alkylamino, arylamino, amido, nitro, thiol, alkylthio, arylthio, alkylsulfonyl, carbamate, and urea, or two of said substituents may be joined together to form a fused five-, six-, or seven-membered carbocyclic or heterocyclic ring comprising zero to three heteroatoms;
to a patient, wherein the effect is selected from the group consisting of improving glucose tolerance, decreasing insulin resistance, decreasing body weight, controlling weight gain, modulation of type I diabetes, modulation of type II diabetes, modulation of perturbed bile acid metabolism, modulation of rheumatoid arthritis, modulation of ulcerative colitis, and modulation of inflammatory bowel disease.
